(19) United States
(12) Patent Application Publication
(10) Pub. No.: US 2026/0294624 A1
(43) Pub. Date: Oct. 1, 2026

(54) Ventricular Control of Prosthetic Atrioventricular Valve

(71) Applicant: St. Jude Medical, Cardiology Division, Inc., St. Paul, MN (US)

(72) Inventors: Randolf Von Oepen, Aptos, CA (US); John F. Otte, Minneapolis, MN (US)

(73) Assignee: St. Jude Medical, Cardiology Division, Inc., St. Paul, MN (US)

(21) Appl. No.: 19/480,456

(22) PCT Filed: Apr. 22, 2024
(86) PCT No.: PCT/US2024/025643

§ 371 Date: Oct. 31, 2025

Related U.S. Application Data
(60) Provisional application No. 63/500,993, filed on May 9, 2023.

(51) Int. Cl. A61F 2/24

(52) U.S. Cl. CPC A61F 2/2436; A61F 2/2418; A61F 2/2439; A61F 2230/001

(57) ABSTRACT

According to one aspect of the disclosure, a prosthetic heart valve system includes a collapsible and expandable prosthetic atrioventricular valve including an atrial disk, a ventricular disk, a center portion, and a plurality of prosthetic leaflets. The system may include a delivery device that includes a catheter having a valve cover configured to maintain the prosthetic heart valve in a collapsed condition for delivery. The delivery device may include an expansion restriction mechanism and a shaft system including an atrial tube and a ventricular tube. In a delivery condition, the prosthetic heart valve is collapsed within the valve cover and pluralities of sutures may connect the atrial and ventricular tubes to the atrial and ventricular disks, respectively, to restrict and control the expansion of the ends of the prosthetic heart valve.

### CROSS-REFERENCE TO RELATED APPLICATIONS

This application claims the benefit of priority to U.S. Provisional Patent Application No. 63/500,993, filed on May 9, 2023, the disclosure of which is hereby incorporated by reference herein.

### BACKGROUND OF THE DISCLOSURE

Heart valve disease is a significant cause of morbidity and mortality. One treatment for this disease is valve replacement. One form of replacement device is a bioprosthetic valve. Collapsing these valves to a smaller size or into a delivery system enables less invasive delivery approaches compared to conventional open-chest, open-heart surgery. Collapsing the implant to a smaller size and using a smaller delivery system minimizes the access site size and reduces the number of potential periprocedural complications.

The size to which an implant can be collapsed is limited by the volume of materials used in the implant, the strengths and shapes of those materials, and the need to function after expansion (or re-expansion). Using multiple steps and/or multiple delivery system devices may increase the time and complexity of a procedure.

Native atrioventricular valves (i.e., the tricuspid valve and the mitral valve) typically have a larger size and/or diameter compared to the native aortic valve and the native pulmonary valve. Among the native atrioventricular valves, a regurgitant tricuspid valve typically has a larger size and/or diameter than a regurgitant mitral valve. For example, for patients with severe tricuspid valve regurgitation, the diameter of the tricuspid valve may range from about 30 mm to about 70 mm (including about 40 mm to about 66 mm), although these numbers are merely exemplary. As a result, prosthetic heart valve designs and considerations for replacing the different native heart valves are not identical. For example, to accommodate the large size of the mitral and tricuspid valve, recent prosthetic heart valve designs have included an outer frame with a large size to engage the native mitral or tricuspid annulus, and a smaller and generally cylindrical inner frame within that outer frame, the inner frame housing the prosthetic valve leaflets. However, this double-stented design generally increases the bulk of the prosthetic heart valve, resulting in a larger profile when collapsed within a delivery device. This, in turn, requires the delivery device (e.g., a catheter housing the collapsed prosthetic heart valve for delivery) to have a larger size to accommodate the large prosthetic heart valve. Typically, it is desirable for catheters of transcatheter heart valve delivery devices to have a smaller size, since the catheters may need to pass through the vasculature to reach the native heart valve in a minimally invasive manner.

When delivering a collapsible and expandable prosthetic atrioventricular valve via a fully intravascular approach (e.g. via the femoral vein and inferior vena cava), a portion of the tip of the delivery device housing or otherwise containing the prosthetic heart valve typically needs to be at least partially positioned within the ventricle to properly align with the native valve annulus. For example, with some self-expanding prosthetic mitral valves, prior to expansion, about one-third of the length of the collapsed prosthetic mitral valve is arranged on the atrial side of the mitral valve annulus and about two-thirds of the length of the collapsed prosthetic mitral valve is arranged on the ventricular side of the mitral valve annulus. This may require a significant space for the delivery device to be positioned within the left ventricle for a successful prosthetic valve implantation. While this spacing may make implantations difficult in at least some circumstances, prosthetic tricuspid valves—particularly those with a single support frame—may have a large length when collapsed. Further, the right ventricle is often shorter in available length compared to the left ventricle. Thus, prosthetic tricuspid valve implantations may be particularly difficult, although it should be understood that the disclosure herein may be applicable to both mitral and tricuspid valve implants and procedures. However, it should be understood that the device and methods described herein to allow for better control and/or release of a self-expanding prosthetic heart valve during deployment may apply to heart valve replacement, including the aortic valve, pulmonary valve, mitral valve, and tricuspid valve, and may even apply to any other self-expanding device that is delivered intravascularly which would benefit from a more controlled deployment.

### SUMMARY OF THE DISCLOSURE

According to one aspect of the disclosure, a prosthetic heart valve system includes a prosthetic heart valve for replacing a native atrioventricular valve. The prosthetic heart valve may include a collapsible and expandable frame that includes an atrial disk, a ventricular disk, and a center portion extending between the atrial disk and the ventricular disk, and a plurality of prosthetic leaflets disposed within the frame. The system may include a delivery device for delivering and deploying the prosthetic heart valve, which may include a catheter having a valve cover at a distal end thereof, the valve cover configured to maintain the prosthetic heart valve in a collapsed condition for delivery. The delivery device may include an expansion restriction mechanism and a shaft system including an atrial tube and a ventricular tube, the expansion restriction mechanism including a first pair of rings fixed to the atrial tube and a second pair of rings fixed to the ventricular tube. In a delivery condition of the system, the prosthetic heart valve is collapsed within the valve cover with the atrial disk positioned adjacent to the atrial tube and the ventricular disk positioned adjacent to the ventricular tube, and a plurality of first sutures connect the first pair of rings and extend through the atrial disk, and a plurality of second sutures connect the second pair of rings and extend through the ventricular disk.

In a partially deployed condition of the system, (i) the valve cover may be withdrawn relative to the prosthetic heart valve so that the valve cover does not restrict expansion of the prosthetic heart valve; (ii) the plurality of first sutures may be wound around the atrial tube so that an inflow portion of the atrial disk is restricted from self-expanding; (iii) the plurality of second sutures may be wound around the ventricular tube so that an outflow portion of the ventricular disk is restricted from self-expanding; and (iv) an outflow portion of the atrial disk, an inflow portion of the ventricular disk, and the center portion of the frame are all at least partially expanded.

In the partially deployed condition of the system, the prosthetic heart valve may have a peanut shape in which the at least partially expanded inflow portion of the ventricular disk has a larger diameter than the at least partially expanded outflow portion of the atrial disk, which has a larger diameter than the at least partially expended center portion of the frame.

The system may include a gear system coupled to the delivery device, the gear system having a first connector coupled to the atrial tube, a second connector coupled to the ventricular tube, and a knob operatively coupled to the first and second connectors such that rotation of the knob in a first rotation direction first and second connectors, and thus the atrial and ventricular tubes, to simultaneously rotate in opposite directions. The first connector may be directly coupled to the atrial tube, and the second connector may be directly connected to a central rod which passes through the atrial tube and directly connects to the ventricular tube.

The first pair of rings may include a first mounting ring and a first suture ring, and the second pair of rings may include a second mounting ring and a second suture ring, the first plurality of sutures being fixedly coupled to the first mounting ring and releasably coupled to the first suture ring, and the second plurality of sutures being fixedly coupled to the second mounting ring and releasably coupled to the second suture ring. The first suture ring may have a plurality of first fingers that each have a free end pointing in a first rotational direction, and the second suture ring may have a plurality of second fingers that each have a free end pointing in a second rotational direction opposite the first rotational direction.

The first pair of rings may include first and second mounting rings, and the second pair of rings may include third and fourth mounting rings, the first plurality of sutures being fixedly coupled to both the first and second mounting rings, and the second plurality of sutures being fixedly coupled to both the third and fourth mounting rings. The system may include a first pull rod extending through the first pair of rings, eyelets of the first plurality of sutures receiving the first pull rod therethrough in the delivery condition of the system, and a second pull rod extending through the second pair of rings, eyelets of the second plurality of sutures receiving the second pull rod therethrough in the delivery condition of the system, the first and second pull rods each being configured to be withdrawn relative to the respective pair of rings to release the respective eyelets from the respective pull rod.

The atrial disk may include a plurality of atrial apertures formed at respective inflow tips of the atrial disk, and the ventricular disk may include a plurality of apertures formed at respective outflow tips of the ventricular disk, and in the delivery condition of the system, the plurality of first sutures may pass through respective ones of the atrial apertures, and the plurality of second sutures may pass through respective ones of the ventricular apertures.

According to another aspect of the disclosure, a method of implanting a prosthetic heart valve includes loading the prosthetic heart valve into a delivery device, the prosthetic heart valve including a collapsible and expandable frame having an atrial disk, a ventricular disk, a center portion extending between the atrial disk and the ventricular disk, and a plurality of prosthetic leaflets disposed within the frame. The delivery device may be advanced to a native heart valve of a patient while the prosthetic heart valve is maintained in a collapsed condition by a valve cover of the delivery device. While the delivery device is positioned in or adjacent to the native heart valve, the prosthetic heart valve may be started to be deployed by withdrawing the valve cover so that the frame begins to self-expand. As the frame begins to self-expand, an outflow end portion of the ventricular disk may be restricted from self-expanding due to a connection between the outflow end portion of the ventricular disk and a ventricular tube positioned inside the frame, such that the ventricular disk is partially expanded after the valve cover is withdrawn. After the valve cover is withdrawn and the ventricular disk is partially expanded, a desired position of the prosthetic heart valve relative to the native heart valve may be confirmed. After confirming the desired position of the prosthetic heart valve relative to the native heart valve, the connection between the outflow end portion of the ventricular disk and the ventricular tube may be released to allow the ventricular disk to expand into engagement with the native heart valve.

As the frame begins to self-expand, an inflow end portion of the atrial disk may be restricted from self-expanding due to a connection between the inflow end portion of the atrial disk and an atrial tube positioned inside the frame, such that the atrial disk is partially expanded after the valve cover is withdrawn. After the valve cover is withdrawn, but while the connection between the outflow end portion of the ventricular disk and the ventricular tube is maintained and while the connection between the inflow end portion of the atrial disk and the atrial tube is maintained, the prosthetic heart valve may have a peanut shape in which an outflow portion of the atrial disk is at least partially expanded and an inflow portion of the ventricular disk is at least partially expanded. When the prosthetic heart valve has the peanut shape, the at least partially expanded atrial disk may have a first diameter, the at least partially expanded ventricular disk may have a second diameter, and the center portion of the frame may have a third diameter, the second diameter being larger than the first diameter, and the first diameter being larger than the third diameter.

After confirming the desired position of the prosthetic heart valve relative to the native heart valve, the connection between the inflow end portion of the atrial disk and the atrial tube may be released to allow the atrial disk to expand into engagement with the native heart valve. The connection between the outflow end portion of the ventricular disk and the ventricular tube may be formed by a first plurality of sutures, and the connection between the inflow end portion of the atrial disk and the atrial tube may be formed by a second plurality of sutures. In the partially expanded condition of the ventricular disk, the first plurality of sutures may be wound around the ventricular tube, and in the partially expanded condition of the atrial disk, the second plurality of sutures may be wound around the atrial tube. Releasing the connection between the outflow end portion of the ventricular disk and the ventricular tube may include allowing the first plurality of sutures to unwind from the ventricular tube, and releasing the connection between the inflow end portion of the atrial disk and the atrial tube may include allowing the second plurality of sutures to unwind from the atrial tube. Allowing the first plurality of sutures to unwind from the ventricular tube and allowing the second plurality of sutures to unwind from the atrial tube may include simultaneously rotating the atrial tube in a first rotational direction and rotating the ventricular tube in a second rotational direction opposite the first rotational direction. Releasing the connection between the outflow end portion of the ventricular disk and the ventricular tube and releasing the connection between the inflow end portion of the atrial disk and the atrial tube may include either: (i) allowing the first plurality of sutures and the second plurality of sutures to slip off respective connection points to their respective tubes; or (ii) actively withdrawing a first pull rod to disconnect the first plurality of sutures from the ventricular tube and actively withdrawing a second pull rod to disconnect the second plurality of sutures from the atrial tube.

### BRIEF DESCRIPTION OF DRAWINGS

FIG. 1A is a perspective view of a prosthetic atrioventricular valve according to one aspect of the disclosure.

FIG. 1B is a view of a cut pattern of a stent for use with the prosthetic heart valve of FIG. 1A.

FIG. 1C shows the prosthetic heart valve of FIG. 1A with certain structures omitted from the view for clarity.

FIG. 1D is a schematic side view of a portion of a tubular instrument of a delivery device.

FIG. 1E is an enlarged view of an end of the tubular instrument of FIG. 1D.

FIGS. 1F-G are sectional longitudinal views of a tubular assembly of the tubular instrument of FIG. 1D.

FIG. 1H is a side view of FIG. 1E with a wire structure in a radially retracted state.

FIG. 2A illustrates a frame of a prosthetic heart valve in a state of partial expansion, with other components of the prosthetic heart valve being omitted for clarity.

FIG. 2B is a top view of a gear system for use in controlling the partial expansion of the frame of FIG. 2A.

FIG. 2C is a highly schematic illustration of components of the gear system of FIG. 2B coupled to a shaft system and the frame of FIG. 2A.

FIGS. 3A-B are perspective views of expansion control mechanisms that may be used with the system of FIGS. 2A-C.

FIGS. 3C-D illustrate alternate suture loop configurations for use with the expansion control mechanisms of FIGS. 3A-B.

FIGS. 4-5 are perspective views of additional alternate control mechanisms with features in common to the expansion control mechanisms of FIGS. 3A-B.

FIG. 6A is a view of a portion of a cut pattern of a stent for use with the prosthetic heart valve of FIG. 1A.

FIG. 6B is an enlarged view of a ventricular cell of the frame of FIG. 6A with a tine control mechanism in use.

FIGS. 7A-D are highly schematic illustrations of different steps of implanting a prosthetic tricuspid valve using expansion control mechanisms of the disclosure.

### DETAILED DESCRIPTION OF THE DISCLOSURE

As used herein, the term inflow, when used in connection with a prosthetic heart valve, refers to the end of the prosthetic heart valve through which blood first flows when flowing in the antegrade direction, and the term outflow refers to the end of the prosthetic heart valve through which blood last flows when flowing in the antegrade direction. Further, although the disclosure focuses on prosthetic tricuspid valve replacements, the disclosure may apply to prosthetic mitral valve replacements. Thus, unless otherwise expressly specified, the embodiments described herein may be used for replacing either a native tricuspid valve or a native mitral valve (with or without additional modifications specific to the heart valve being replaced), even if a particular embodiment may be more suited for replacing either the native tricuspid valve or the native mitral valve. However, as noted above, it should be understood that the device and methods described herein to allow for better control and/or release of a self-expanding prosthetic heart valve during deployment may apply to heart valve replacement, including the aortic valve, pulmonary valve, mitral valve, and tricuspid valve, and may even apply to any other self-expanding device that is delivered intravascularly which would benefit from a more controlled deployment.

As explained in the background of the disclosure, prosthetic heart valves that include an anchoring frame and a valve frame nested within the anchoring frame typically have larger sizes when collapsed within a delivery device. As an example, this type of prosthetic heart valve may only fit within a delivery device that has a catheter with an outer diameter that is 24-40 French (8-13.33 mm in diameter) or larger, including 30-33 French (10-11 mm in diameter) or larger. For transcatheter prosthetic mitral or tricuspid valves that are delivered intravascularly through the femoral vein, a delivery catheter having an outer diameter of 30-33 French or larger may increase the likelihood of access site complications, which may require a vascular surgeon to intervene. The prosthetic heart valves disclosed herein have features and configurations that are intended to allow for the prosthetic heart valves to reliably anchor within the larger size annulus of the tricuspid valve (or the mitral valve) while being able to collapse into a delivery catheter having an inner diameter of 30-33 French or smaller, including as small as 24 French (8 mm). It should be understood that, as used herein, the unit French refers to the inner diameter of a catheter when describing the ability of a valve to fit within that catheter, whereas the unit French refers to the outer diameter of the catheter when describing how catheter size may result in vascular access problems.

As is described below, one way to achieve this functionality is to design the prosthetic heart valve with a single support stent (e.g., a single stent layer or a non-nested frame configuration) that can span the large atrioventricular valve annulus diameters found in patients who experience heart failure. The geometry of the support stent may allow for prosthetic leaflets to be secured inside, with atrial and/or ventricular flanges or disks that have a large enough diameter or profile to sandwich, clamp, or overlie the native annulus tissue therebetween. To provide adequate sealing between the support stent and the native valve annulus, fabric(s) may span the gap between the atrial and ventricular disks, where the fabric(s) are capable of elongating to mitigate the effects of lengthening when sheathing the prosthetic heart valve into the catheter. While the embodiments described below may be suitable for replacing either a tricuspid or mitral valve, these embodiments may be best suited for replacing a tricuspid valve due to the lower right ventricular pressures compared to left ventricular pressures, which may reduce the need for a nested stent design. It should be understood that, although the terms “frame” and “stent” are generally used interchangeably herein, the term “stent” does not imply any special structure or function beyond being a frame.

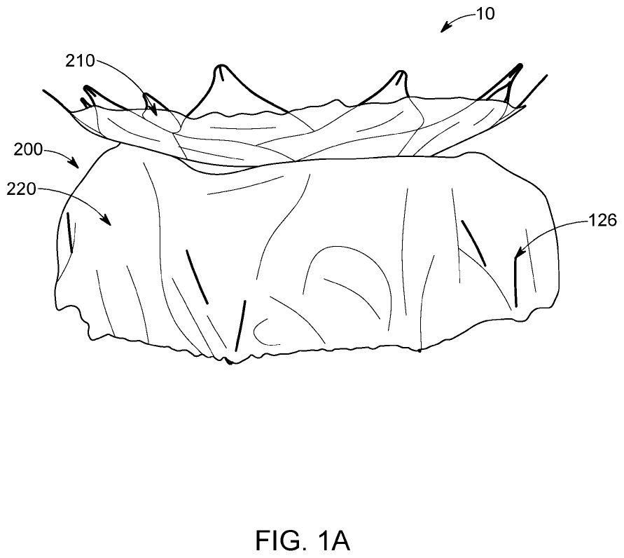

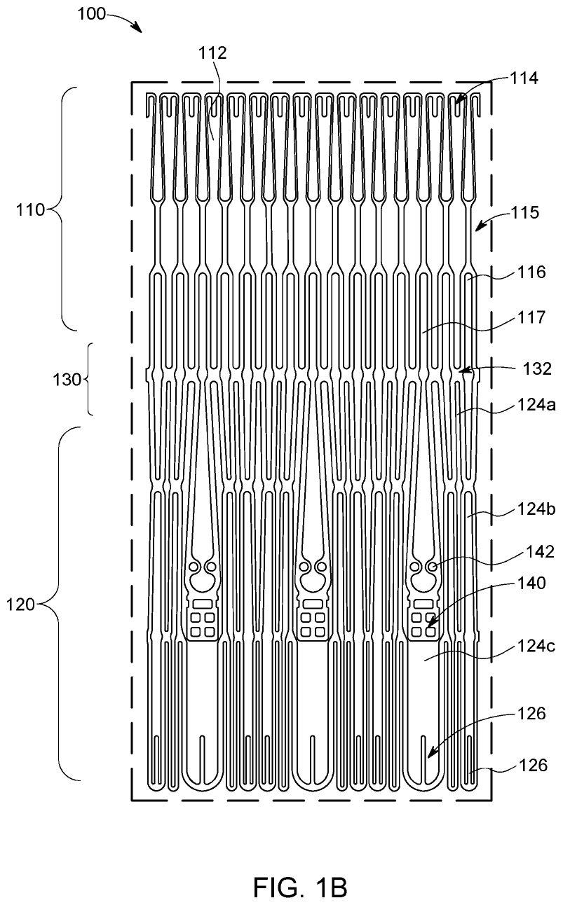

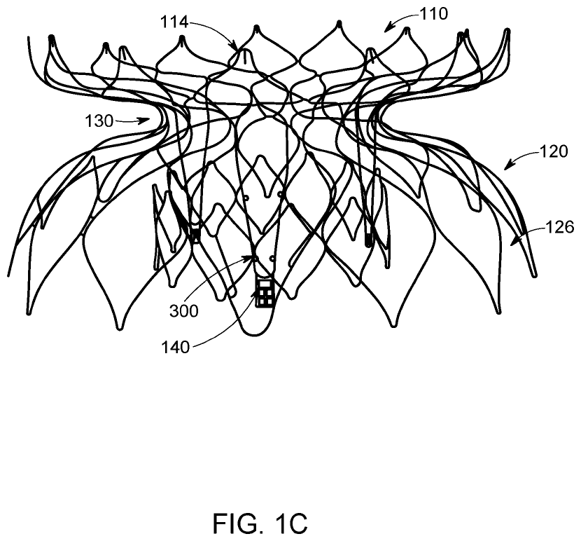

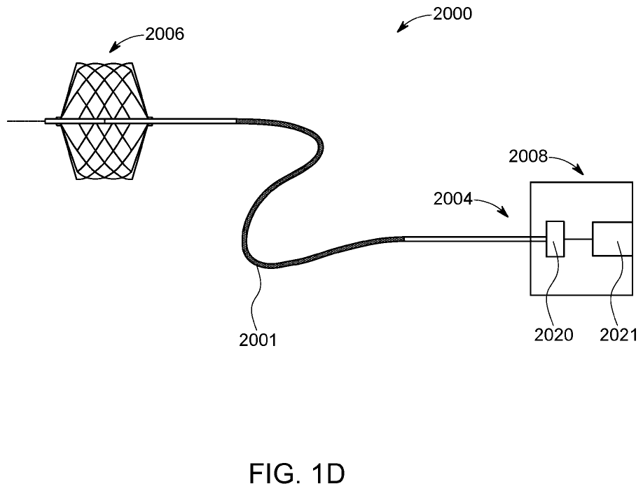

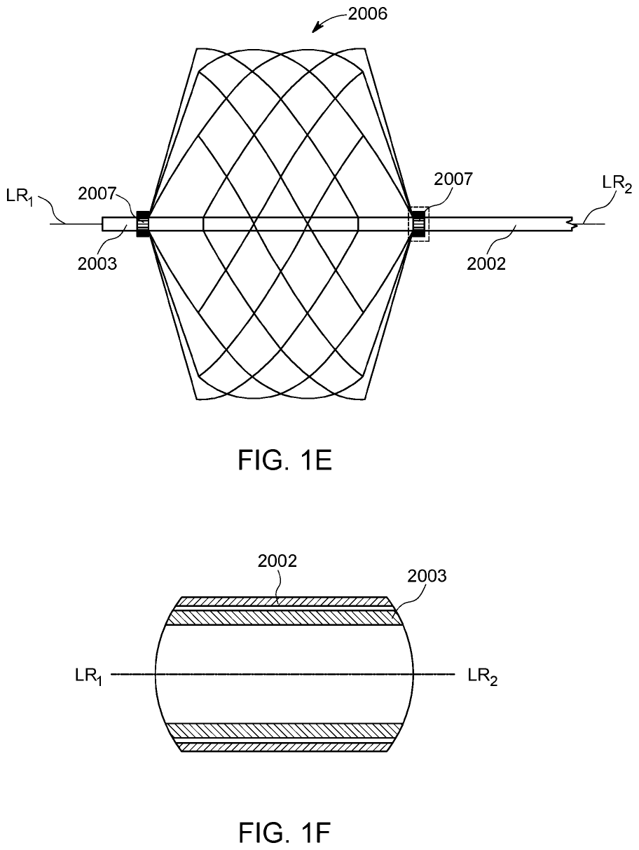

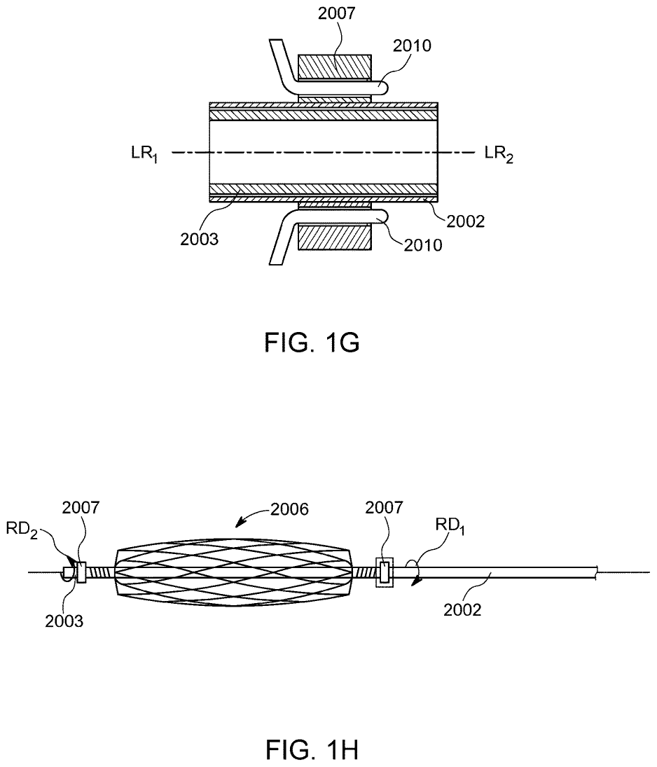

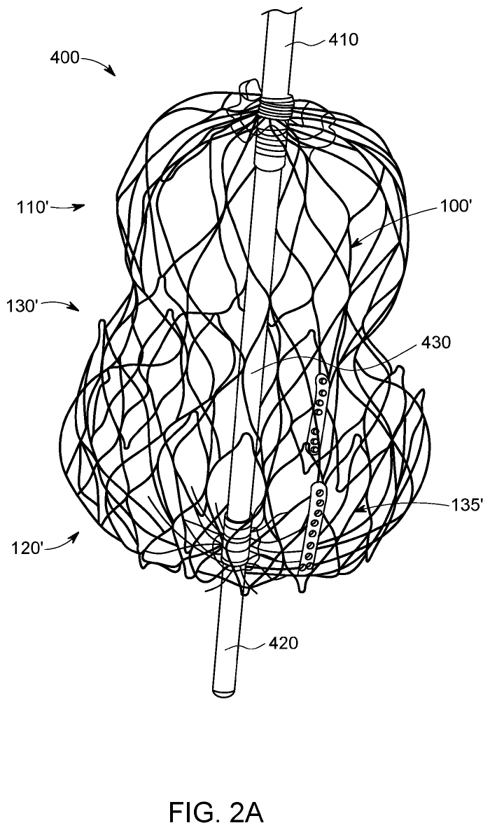

FIG. 1A illustrates a prosthetic heart valve 10 according to an embodiment of the disclosure. Prosthetic heart valve 10 may be particularly suited for replacing a native atrioventricular valve, and in particular the native tricuspid valve. Prosthetic heart valve 10 may include four components generally, including a stent or frame 100, a sealing skirt 200, prosthetic leaflets (not visible in FIG. 1A), and a commissure support member 300 (shown in FIG. 1C), which may also be referred to as a stiffening structure. Other components may be provided in addition to these four components, such as sutures to stitch the fabric (e.g. sealing skirt 200) and/or tissue (e.g., prosthetic leaflets) to the frame 100 and/or commissure support member 300. Prosthetic heart valve 10 is shown in FIG. 1A in an expanded or deployed condition and is oriented with the atrial or inflow end of the valve toward the top of the view of FIG. 1A.

FIG. 1B illustrates a portion of a cut pattern of a stent of frame 100 that may be used with prosthetic heart valve 10. In FIG. 1B, frame 100 has the same orientation as shown in FIG. 1A. In other words, in the view of FIG. 1B, the inflow or atrial end of frame 100 is oriented toward the top of the view. Frame 100 is preferably formed from a shape-memory material, such as a nickel-titanium alloy such as Nitinol, and may be created from a single tube, for example via laser cutting a tube of Nitinol. In the cut patterns shown in FIG. 1B, frame 100 generally includes an atrial portion 110 and a ventricular portion 120 separated by a center portion 130. After frame 100 is cut and set to the desired shape, for example as shown and described in greater detail in connection with FIG. 1C, the center portion 130 may be very short.

Frame 100 may include an atrial-most or inflow-most row of atrial cells 112, which may be generally diamond-shaped cells that, in the expanded condition, flare radially outwardly from the center portion 130. A pin 114 may be formed at the inflow apex of one, some, or each atrial-most cell 112, the pin 114 extending a short distance in the outflow direction to a free end. Each pin 114 may be sized and shaped so that a suture loop of the delivery device may slip over the pin 114, keeping the frame 100 connected to the delivery device during delivery and deployment. Upon deployment of the prosthetic heart valve 10, each suture loop may be pushed forward or distally to disengage with the corresponding pins 114 to fully decouple the prosthetic heart valve 10 from the delivery device. Similar pins and suture loops are described in more detail in U.S. Pat. No. 10,874,512, the disclosure of which is hereby incorporated by reference herein. The atrial cells 112 may terminate, at their outflow ends, at an inflection point 132. When the frame 100 is shape-set to the desired shape, which may be generally similar to that shown in FIG. 1C, the inflection points 132 may define the smallest diameter of the center portion 130. It should be understood that the term “inflection point” is not necessarily used according to its mathematical definition, but rather references the point at which the frame 100 changes from decreasing diameter to increasing diameter.

Still referring to FIG. 1B, a plurality of transition cells 116, which may be generally diamond-shaped, may be positioned in a row that is adjacent to the atrial cells 112 in the outflow direction. Transition cells 116 may include an inflow portion on the inflow side of center portion 130 and an outflow portion on the outflow side of center portion 130. In some examples, the transition cells 116 may be axially centered about the inflection point 132. The row of transition cells 116 may include three enlarged transition cells 117 (or more or fewer than three depending on the number of prosthetic leaflets included in the prosthetic heart valve 10) that terminate in a commissure attachment feature (“CAF”) 140. Preferably, the enlarged transition cells 117 are positioned at substantially equal circumferential intervals around the frame 100. The sides of the atrial cells 112 (which may extend to the inflow apex of the transition cells 116 and the enlarged transition cells 117) may include elongated beams 115. These elongated beams 115 may provide additional flexibility to the atrial portion 110 (which may be referred to as the atrial disk). For example, depending on the number of cells included in the atrial portion 110, and the desired diameter that the atrial portion will span, the length of the beams 115 may be adjusted. As the desired diameter of the atrial portion 110 increases, the length (in the axial direction) of the diamond-shaped cells that form the atrial portion 110 may need to increase if a particular opening angle (e.g., about 90 degrees) of the diamond-shaped cells is desired. As the axial length of the diamond-shaped cells increases in the differently-sized valve frames, the beams 115 may correspondingly increase or decrease in length. However, in some embodiments, the beams 115 may be omitted and the atrial row of cells 112 may all be “full” diamond-shaped cells.

Each CAF 140 may serve as an attachment point to the prosthetic leaflets. For example, each CAF 140 may include a plurality of holes, and sutures may be used to couple adjacent pairs of leaflets to the CAFs 140 via the holes therein. While CAFs 140 are shown with four small holes in a two-by-two configuration and an elongated hole, other specific CAF configurations may be suitable for use instead of those shown.

The portion of the frame 100 in the outflow direction of the inflection point 132 may include a plurality of ventricular cells. For example, a group of first ventricular cells 124a which may be generally diamond-shaped cells, the inflow apex of which is an inflection point 132. A group of second ventricular cells 124b may extend to the outflow-most portion of the frame 100, the inflow apices of the second ventricular cells being connected to the outflow apices of the transition cells 116. Some, none, or all of the second ventricular cells 124b may include tines 126 that may act as frictional engagement members that frictionally engage native tissue for enhancing securement of the frame 100 within the native valve annulus. A group of third ventricular cells 124c may be positioned between certain pairs of second ventricular cells 124b, and may include struts that extend from the inflection point 132 to the terminal outflow end of the ventricular portion 120. Third ventricular cells 124c may be larger than the other ventricular cells and may be formed in part by the struts of enlarged transition cells 117 that terminate at CAFs 140. With this configuration, the CAFs 140 may be thought of as either nested within third ventricular cells 124c or forming a boundary of third ventricular cells 124c.

In addition to tines 126 being positioned in some, none, or all of the second ventricular cells 124b, none, some, or all of the third ventricular cells 124c may include tines 126. In the illustrated embodiment, each third ventricular cell 124c includes a single tine 126 extending upward from an outflow apex of the cell, and only some of the second ventricular cells 124b include tines 126, such that each second ventricular cell 124b that includes a tine 126 includes a single tine 126 extending upward from an outflow apex of the cell. All of the tines 126 may extend to a free tip that may have a sharp or blunt point, that is intended either to pierce tissue or to frictionally engage the tissue without piercing it. It should be understood that the number and positioning of the tines 126 may be different from those shown in FIG. 1B, and the specific number and positioning shown in FIG. 1B.

In the illustrated embodiment, the tines 126 may be connected at an outflow end of the tine, with the free tip being positioned at an inflow end of the tine. This directionality of tines, compared to the tines being connected at their inflow end and having free tips at their outflow ends, may allow for a smoother and easier deployment of the valve from the delivery catheter. In other words, as the valve begins to self-expand as it is released from the delivery catheter, the tines do not begin to expand until the entire tine is free of the delivery device. With the opposite orientation, the tines might otherwise begin to extend radially outwardly and into contact with the end of the delivery sheath, which might make deployment more difficult. However, it should be understood that the illustrated directionality of tines may make the loading process slightly more difficult compared to the opposite directionality. However, smooth and easy deployment is typically more important than smooth and easy loading, and the loading process can be highly controlled and is performed outside the patient, while the deployment process is performed inside the patient.

After forming the frame 100 by using the cut pattern shown in FIG. 1B, or another generally similar cut pattern, the frame 100 may be shape-set, for example via heat treatment, to the desired shape. FIG. 1C illustrates one example of a frame 100 that has a cut pattern similar to that shown in FIG. 1A, after having been shape set and having been connected to a commissure support 300, described in greater detail below.

As can be seen in FIG. 1C, when the frame 100 is in the expanded or deployed condition the bottom of the atrial portion 110 may be substantially straight with a slight upward angle, while the top half of the atrial portion 110 may flare upwardly so that the tips of the atrial cells 112 point generally in the inflow direction. The contours described above may be other than as exactly described while still being suitable for use in the prosthetic heart valve 10.

Still referring to FIG. 1C, the ventricular portion 120 may form a general “bell” shape with a more rounded and less flat contour compared to the atrial portion 110. The more gentle contour of the ventricular portion 120 may allow for the ventricular portion 120 to drape against the ventricle and apply only light pressure to assist in fixing or otherwise securing the prosthetic heart valve 10 to the native valve annulus. This light pressure or draping may be a first mechanism by which the prosthetic heart valve 10 achieves fixation within the native valve annulus.

The various tines 126 described above may be shape set so that the free ends of the tines 126 are positioned away from the surfaces defined by the cell in which the tine 126 is located. In other words, the tines 126 may be bent or shaped so that the tips are available to pierce tissue or to frictionally engage tissue without piercing to provide a second mechanism by which the prosthetic heart valve 10 achieves fixation within the native valve annulus. The tines 126 may be oriented at different angles to achieve different objectives. For example, in some embodiments, some or all of the tines 126 may be oriented or angled with the free ends pointing toward the atrial portion 110 at an acute angle relative to the longitudinal axis passing through the center of the prosthetic heart valve 10. Tines 126 pointing at an acute angle, compared to a right angle or an obtuse angle, may be less likely to perforate tissue at the native valve annulus. Patients that may be in need of a prosthetic atrioventricular valve, particularly a prosthetic tricuspid valve, may be likely to have very thin medial walls in the ventricle, and acutely angled tines 126 may particularly reduce the likelihood of the medial wall getting perforated by the tines 126. There may be additional benefits to having an acutely angled tine 126 compared to tines 126 with larger angles (e.g., right angle or obtuse angle), relating to loading and deployment of the prosthetic heart valve 10. For example, if the tines 26 are more acutely angled, they may provide less resistance when the prosthetic heart valve 10 is loaded into, or deployed from, the delivery catheter. Less resistance may equate to a more manageable load, which—all else being equal—may allow for a smaller size delivery catheter to be used. However, this is just one option. Some or all of the tines 126 may instead be shape-set to be oriented more laterally, for example a relatively large acute angle, or a right or obtuse angle, relative to the central longitudinal axis of the prosthetic heart valve 10. Although the tines 126 may be optional entirely, if the tines 126 are included, whether they are acutely or laterally oriented, the tines 126 may provide a second mechanism by which the prosthetic heart valve 10 achieves fixation within the native valve annulus.

Before describing the support member 300 in more detail, an exemplary sealing skirt 200 that may be used with the prosthetic heart valve 10 is described. Referring to FIG. 1A, an outer sealing skirt 200 may be provided on the exterior of the frame 100. In some embodiments, the sealing skirt 200 may be the same as or similar to any of the embodiments described in U.S. Provisional Patent Application No. 63/384,521, filed Nov. 21, 2022, and titled “Transcatheter Prosthetic Atrioventricular Valve with Stiffening Structure,” the disclosure of which is hereby incorporated by reference herein. In the particular example shown in FIG. 1A, the sealing skirt 200 may be a single piece of material (although in some embodiments it may be a multi-piece design), which may be formed from any suitable material, such as polyethylene terephthalate (“PET”), polytetrafluoroethylene (“PTFE”), ultra-high molecular weight polyethylene (“UHMWPE”), polyester, or similar materials or combinations of such materials). It may be preferable for the sealing skirt 200 to be formed as a woven skirt, but other options, including forming as a knitted skirt, may be suitable in some embodiments. In the illustrated embodiment of FIG. 1A, the sealing skirt 200 is formed from woven PET and may have an atrial skirt portion 210 and a ventricular skirt portion 220. The atrial skirt portion 210 may be coupled to the atrial portion 110 of the frame 100 with a relatively tight connection—for example via suturing along the struts of the atrial portion 110 of the frame 100. In some embodiments, including that shown in FIG. 1A, the inflow edge of the atrial skirt portion 210 may be positioned a spaced distance from the atrial tips of the atrial cells 112. For example, in some embodiments that atrial end of the frame 100 inflects towards the atrium (not shown in FIG. 1A), and thus outer sealing skirt 200 may be terminated a spaced distance from the atrial end so that there is not a thrombogenic profile on the inflow end of the frame 100. As used herein, the term “thrombogenic profile” refers to a shape or profile that may promote stagnation of blood. In other embodiments, the inflow edge of the atrial skirt portion 210 may be positioned to align with or cover the atrial tips of the atrial cells 112. It should be understood that the various tines 126 preferably pierce through the sealing fabric 200 so that the free ends thereof are available for frictional engagement with the native tissue upon implantation.

Still referring to FIG. 1A, the ventricular skirt portion 220 may be more loosely connected to the ventricular portion 120 of the frame 100 than the atrial skirt portion 210 is connected to the atrial portion 110. For example, the outflow edge of the ventricular skirt portion 220 may be relatively tightly coupled to the outflow end of the ventricular portion 120 of the frame 100, but the connection of the sealing skirt 200 may be relatively loose between the central portion 130 and the terminal end of the ventricular portion 120 of the frame 100. With this configuration, during ventricular systole (e.g., as the ventricle contracts, the prosthetic leaflets close, and the pressure in the ventricle is greater than the pressure in the atrium), the pressure differential causes the ventricular skirt portion 220 to billow, inflate, or parachute open. As the ventricular skirt portion 220 parachutes during ventricular systole, it may fill any gaps, crevices, or openings between the prosthetic heart valve 10 and the native valve annulus that might otherwise result in blood leaking around the outside of the prosthetic heart valve 10 back into the atrium (i.e., paravalvular or “PV” leak). Typically, sealing of a self-expanding prosthetic heart valve results, at least in part, from “oversizing” the prosthesis relative to the annulus to ensure that the prosthetic heart valve presses against the annulus with force to help sealing. However, particularly for the tricuspid valve, it may be desirable to avoid over-stretching the native annulus, which may occur if the prosthesis is over-sized. Achieving sealing with this billowing skirt action may allow the prosthesis to not be, or not significantly be, oversized relative to the native tricuspid annulus.

Referring briefly to FIG. 1C, the illustrated configuration of frame 100 may provide a levering effect that may further assist with sealing against PV leak. For example, when the frame 100 is in the expanded or deployed state shown in FIG. 1C, deformation of the ventricular portion 120 may tend to lever the atrial portion 110 toward the ventricular portion 120. Thus, referring back to FIG. 1A, as the ventricular skirt portion 220 inflates or parachutes during ventricular systole, which may cause the ventricular portion 120 of the frame 100 to slightly deform, the atrial portion 110 of the frame 100 may be lightly pulled downward against the atrial side of the native valve annulus. This “sandwiching” action may further seal against any PV leak, and may also mitigate potential embolization. For example, particularly in the low flow environment of the right heart, any gaps or spaces left between the prosthetic heart valve 10 and the native anatomy may create a thrombus risk zone. Further, any gaps or spaces create an increased risk of PV leak, which may tend to accelerate the blood flowing through the gap space, causing additional shear stress on red blood cells and therefore damage said cells. The above-described levering or sandwiching effect may reduce or eliminate any such gaps or spaces, thus reducing the risk of thrombus formation. In one particular example, patients may have a pronounced septal bump, and some patients may have in particular a septal bump in the right ventricle that overhands the tricuspid valve annulus. This anatomy may be an exclusion criterion for a transcatheter prosthetic tricuspid valve replacement. However, the sandwiching or levering effect described above may allow for prosthetic heart valve 10 to be implanted into patients who have relatively pronounced septal bumps.

Referring again to FIG. 1C, in the deployed or expanded condition of the frame 100, the bottom struts of the enlarged transition cells 117, to which the CAFs 140 are connected, extend in the outflow direction substantially parallel to the central longitudinal axis of the prosthetic heart valve 10. With this positioning, the CAFs 140 may be positioned in alignment with, or nearly in alignment with, the smallest diameter portion of the frame 100 at the central potion 130. In other words, the CAFs 140 of frame 100 are effectively cantilevered. This cantilevering of the CAFs 140, if no additional support is provided, may result in certain disadvantages. As explained above, the prosthetic leaflets are coupled to the CAFs 140. As a result, during ventricular systole when the prosthetic leaflets are closed and pressure is applied in the ventricular-to-atrial direction, the CAFs 140 and the struts of the enlarged transition cells 117 to which the CAFs 140 are attached may deflect radially inwardly toward each other. Although some amount of deflection may be desirable, the length of the CAFs 140 (which may extend between about 20-30 mm from the central portion 130) may be such that a risk of over-deflection may result. If the CAFs 140 deflect too much during ventricular systole, the prosthetic leaflets may not coapt correctly, leading to inefficient valve functionality. Also, another disadvantage of large amounts of deflection of the CAFs 140 is that the struts from which the CAFs 140 extend may fatigue rapidly, possibly leading to failure of the frame 100.

Other potential disadvantages may result if the CAFs 140 of frame 100 do not have additional support. For example, during deployment of prosthetic heart valve 10, the ventricular or outflow end of the prosthetic heart valve 10 may tend to overexpand while the atrial or inflow end of the prosthetic heart valve 10 remains collapsed within the delivery device. This overexpansion or splaying may stress the prosthetic leaflets coupled to the frame 100. The commissure support 300 described below may help prevent such overexpansion or splaying.

In order to address any one or more of the potential disadvantages of CAFs 140 that exclude additional support members, a commissure support member 300 (which may be referred to herein as a CAF support or simply a support member) may be provided. The CAF support 300 is shown assembled to the frame 100 in FIG. 1C. The CAF support 300 may take various forms, but in some examples it may be an expandable and collapsible ring-shaped structure. In the illustrated embodiment, the CAF support 300 is formed of a shape-memory material, such as Nitinol, and may be laser-cut from a Nitinol tube using a diamond-shaped pattern similar to that shown in FIG. 1C. After cutting the CAF support 300, the resulting structure may be shape set (e.g., via heat treatment) so that, in the absence of applied forces, the CAF support 300 forms a generally circular or cylindrical ring having a single row of diamond-shaped cells. In the expanded or unbiased condition, the interior diameter of CAF support 300 is about equal to the diameter of a circle that is aligned with the outer surfaces of the CAFs 140 when the frame 1100 is in its expanded or unbiased condition.

The CAF support 300 may be positioned on the exterior of the CAFs 140 (and/or the cell struts from which the CAFs 140 extend) and coupled to the frame 100 via any suitable mechanism. For example, in some embodiments, the CAF support 300 may be simply sutured to the CAFs 140 and/or to the cell struts from which the CAFs 140 extend. In other embodiments, either the CAF support 300, the CAFs 140 (or their associated struts), or both may include features to assist in the fixation. For example, referring back to FIG. 1B, one or both of the two struts that lead to the CAF 140 may include one or more apertures 142 that may be used to assist suturing the CAF support 300 (e.g., at an intersection where two adjacent diamond-shaped cells of CAF support 300 meet) to the frame 100. In the frame 100 shown in FIG. 1B, each strut leading to a CAF 140 includes a single aperture at the same axial position. However, in the example shown in FIG. 1C, each strut leading to each CAF 140 includes two apertures, and the apertures on one strut may or may not be axially aligned with the apertures on the other strut of the pair. It should be understood that the number, shape, and positioning of apertures 142 may be other than that shown in the figures while still providing the desired functionality. And although suturing is described as one mechanism of fastening the CAF support 300 to the frame 100, it should be understood that other methods, such as adhesives, rivets (or other mechanical fasteners), etc. may be similarly suitable. Further, other designs of CAF support 300 are described in U.S. Provisional Patent Application No. 63/384,521, filed Nov. 21, 2022 and titled “Transcatheter Prosthetic Atrioventricular Valve with Stiffening Structure,” the disclosure of which is hereby incorporated by reference herein. The CAF supports disclosed in that application may be used in connection with the disclosure herein. It should be understood that the CAF support 300 may not be necessary for use with prosthetic heart valve 10, but it may provide benefits if used, including providing better support to the prosthetic heart valve 10 without adding significant additional bulk to the valve.

Prosthetic heart valve 10 may be provided in different sizes to treat different patients. In some examples, the largest size prosthetic heart valve 10 may include atrial disk 110 and/or a ventricular disk 120 having a diameter of around 75 mm in the expanded condition, with a resulting length of between about 40 mm and about 70 mm (including up to about 45 mm, about 50 mm, about 55 mm, about 60 mm, or about 65 mm) when the prosthetic heart valve 10 is in the crimped or collapsed condition within a delivery device. In some embodiments, the delivery device may include a distal tip (e.g. an atraumatic nosecone) having a length of between about 15 mm and about 20 mm. Thus, if it is desirable to have about two-thirds of the collapsed prosthetic heart valve within the ventricle prior to deployment, as described above, a total of up to about 65 mm of length may be required in the ventricle (about 44 mm of collapsed valve length plus up to 20 mm length of nosecone). At least some patients may have a ventricle, in particular a right ventricle, that would simply be too short to accommodate this amount of length. It should be understood that the diameters (in the expanded condition) and lengths (in the collapsed condition) of the prosthetic heart valve 10 (and portions thereof) described above are merely exemplary, and even if the specific dimensions are different than the specific examples listed above, the invention described herein may still provide a similar benefit. However, it should be understood that as the collapsed length of the prosthetic heart valve increases, the desirability for the ventricular control described herein may correspondingly increase.

One option to overcome this potential sizing issue, while still using a similar or identical prosthetic heart valve 10 and delivery device, is to more precisely control how the prosthetic heart valve 10, and (at least in some cases) particularly the ventricular end of the prosthetic heart valve 10 deploys upon an overlying sheath (which may alternatively be referred to as a capsule or valve cover) of the delivery device being withdrawn and removing the constraint keeping the prosthetic heart valve 10 fully collapsed. For example, as is described in greater detail below, the ventricular and atrial ends of the prosthetic heart valve 10 may be separately restricted from expansion via a mechanism other than the valve cover, such that when the valve cover is withdrawn, the prosthetic heart valve 10 will only be able to partially expand due to the additional expansion restriction mechanisms. As this partial expansion occurs, the prosthetic heart valve 10 may expand to have a “peanut” shape (or a double-lobed shape or a shape of conjoined partial spheres). While the prosthetic heart valve 10 has this peanut shape, the ventricular portion of the valve may expand up to a diameter of about 25 mm to 35 mm, without being able to expand completely due to the additional mechanisms restricting the prosthetic heart valve 10 from expanding. Due to the partial radial expansion, the prosthetic heart valve 10 will also partially foreshorten, such that the axial length of the prosthetic heart valve 10 is shorter than the full crimped or collapsed length of the prosthetic heart valve 10. Testing has shown that this type of restriction mechanism allows prosthetic heart valve 10 (and similar designs) to be properly deployed into the native annulus without needing to be positioned as far into the ventricle compared to if prosthetic heart valve 10 had no additional mechanism (besides the valve cover) to restrict its self-expansion. Further, by allowing partial expansion, it may be significantly easier to assess and confirm the desired position of the prosthetic heart valve relative to the native valve annulus prior to actually fully deploying the prosthetic heart valve 10. This may, in turn, allow for easier adjustments and repositioning of the prosthetic heart valve 10 prior to complete deployment. In fact, the prosthetic heart valve 10 could be partially or potentially fully re-collapsed using the mechanisms described herein and repositioned and redeployed to achieve the desired final positioning of the prosthetic heart valve 10. The particular mechanisms described herein may provide ways to safely disconnect the prosthetic heart valve 10 from the delivery device once positioning has been confirmed and/or to adjust the position of the prosthetic heart valve 10 even after one or more tines 126 are exposed.

FIG. 1D is a schematic side view of a tubular instrument 2000 of a delivery device that allows for controlled expansion of a self-expanding device, such as a self-expanding prosthetic heart valve, represented in FIG. 1D by a wire structure 2006. Although referred to as a “wire structure,” it should be understood that this term encompasses not only braided-style stents/frames, but also stents/frames that are laser-cut from a tube (e.g. a hypotube) and any other style of expandable and collapsible stent/frame. FIG. 1E is an enlarged view of a distal end of the tubular instrument 2000 of FIG. 1D. Additional details of a system similar to that shown in FIG. 1D are described in greater detail in International Patent Application Publication No. WO2022/029111, the disclosure of which is hereby incorporated by reference herein. The tubular instrument 2000 may include a tubular assembly 2001 including a first handling tube 2002, a second handling tube 2003, and one or more holding devices 2007. The two handling tubes 2002, 2003 extend from a proximal operating area 2004.

In the illustrated embodiment, the handling tubes 2002, 2003 are arranged coaxially, i.e., their longitudinal axes LR₁ and LR₂ respectively coincide to form a common longitudinal axis of the tubular assembly 2001. The tubular assembly 2001, including handling tubes 2002, 2003, preferably have a desired flexibility or bendability and may be made of any flexible tube material known to the person skilled in the art for intravascular delivery purposes. It should be understood that, although only a portion of tubular assembly 2001 is illustrated to designate flexibility, most or all of the tubular assembly 2001 between the control interface 2008 and the connection point(s) to the wire structure 2006 (both described in more detail below) may have the desired flexibility.

The handling tubes 2002, 2003 are arranged to be relatively rotatable with respect to each other. In particular, in the examples shown, they can be changed in their relative rotational position with respect to the common longitudinal axis of the tubular assembly 2001. At the proximal operating area 2004, the tubular instrument 2000 has a control interface 2008 for relative rotational motion actuation of the first and second handling tubes 2002, 2003. The control interface 2008 can be of desired configuration to achieve the desired rotation and is therefore only shown schematically in FIG. 1D. For example, the control interface 2008 may include a gearbox 2020 for driving rotation of both handling tubes 2002, 2003 and an operating control 2021 that activate the gearbox 2020, for example via motorized actuation or manual actuation. The two handling tubes 2002, 2003 and the control interface 2008 are configured so that only the first or only the second or both handling tubes 2002, 2003 is/are actively rotated, preferably in opposite directions, around the respective longitudinal axis LR₁ and LR₂ (or the common longitudinal axis) in order to achieve the desired relative rotation.

As shown in FIG. 1F, the first handling tube 2002 may overlie the second handling tube 2003. The first handling tube 2002 may be fixedly (e.g. axially and rotationally) coupled to the proximal holding device 2007 (toward the right in the view of FIG. 1E) and the second handling tube 2003 may be fixedly coupled to the distal holding device 2007 (toward the left in the view of FIG. 1E). The holding devices 2007 are configured to hold the wire structure 2006 on the distal area of the tubular assembly 2001. As shown in FIG. 1G, which shows the proximal holding device 2007, a proximal end the wire structure 2006 may be fixedly coupled to the proximal holding device 2007. For example, wires of the wire structure may be secured within recesses of the holding device 2007. In other embodiments, connectors 2010 (e.g. sutures) may be secured within the recesses of the holding device 2007, with other ends of the connectors 2010 coupled to the wire structure 2006. It should be understood that the distal holding structure 2007 may generally be a mirror image of what is shown in FIG. 1G, except that the distal holding structure 2007 is fixedly connected to the second handling tube 2003 instead of the first handling tube 2002.

In some examples, the first handling tube 2002, which may be an outer tube, is formed as a wound coil (or a stack-up of coils wound in opposite directions) of that defines an open center channel. For example, the outer tube may be formed of a first strand wound in the clockwise (or anticlockwise) direction to form a tube, and a strand second wound oppositely in the anticlockwise (or clockwise) direction to form an overlying tube. Although two coils forming tubes are described, more can be provided, including for example a third strand wound in the same direction as the first strand, which forms a tube overlying the second strand. Examples of tubes that may be suitable include Helical Hollow Strand (HHS) offered by Fort Wayne Metals, BiFlex or TriFlex coils offered by Heraeus, Torque Coil offered by Asahi, or other similar products. In some examples, the second handling tube 2003, which may be an inner tube or rod, is formed as a hypotube that is laser cut to impart flexibility, including for example a puzzle cut (which may also be referred to as a ball and socket cut). However, it should be understood that other combinations may be suitable. For example, the inner tube may be formed as the wound torque coils described above, and the outer tube may be formed as the puzzle cut hypotube. Still in other embodiments, both tubes may be formed as the wound torque coils described above, or both may be formed as the puzzle cut hypotubes described above. In practice, however, forming the first handling tube 2002 (or outer tube) as a wound torque coil, and forming the second handling tube 2003 (or inner tube) as a puzzle-cut hypotube, has provided superior results compared to the other options described herein.

Referring now to FIG. 1H, operation of the control interface 2008 causes the first handling tube 2002 to rotate in a first rotation direction RD₁ and the second handling tube 2003 to rotate in a second rotational direction RD₂ opposite the first rotational direction RD₁. This results in two holding devices 2007 rotating in opposite directions, and thus the connectors 2010 to wind around their respective handling tubes 2002, 2003 in opposite direction. This, in turn, will draw the wire structure 2006 inwardly toward the tubular assembly 2001, forcing the first structure to effectively collapse. Operation of the control interface 2008 may also be capable of rotating the handling tubes 2002, 2003 in the opposite directions than shown in FIG. 1H, allowing the connectors 2010 to unwind and allow the wire structure 2006 to begin to self-expand back toward the condition shown in FIG. 1E. With the above described tubular instrument 2000, a self-expanding prosthetic heart valve may be better controlled in terms of expansion and contraction, compared to a self-expanding prosthetic heart valve that is solely controlled via an overlying sheath that is withdrawn to deploy the prosthetic heart valve. However, the above-described tubular instrument 2000 does not address one important feature—the ability to release the prosthetic heart valve from the tubular assembly 2001 for full deployment.

In some embodiments, as the wire structure 2006 radially collapses (i.e. it draws closer to the tubular assembly), it will tend to axially elongate. In some embodiments, it is preferable that, as the wire structure 2006 radially collapses, the holding devices 2007 move away from each other to accommodate the natural tendency of the axial ends of the wire structure 2006 to move away from each other. This can be achieved in various ways, including for example advancing the second handling tube 2003 distally as it rotates, retracting the first handling tube 2002 proximally as it rotates, or both. Although this axial movement could be done manually, more preferably the axial movement is driven via the gearbox 2020 so that a set amount of linear translation may be achieved per set amount of rotation. In other words, by pegging the axial translation of the handling tube(s) to the rotation of the handling tube(s), the exact desired amount of distance between the two holding devices 2007 may always exist depending on the exact amount of rotation that the handling tube(s) have experienced. It should be understood that this movement may work in reverse as well, such that as the handling tubes 2002, 2003 rotate to allow re-expansion of the wire structure 2006, the two holding devices 2007 may draw more closely together as the wire structure 2006 axially foreshortens. This feature may be provided (or omitted) with any of the embodiments described below.

In some embodiments, the outer diameter of the first handling tube 2002 at the point of connection to the corresponding holding device 2007, is identical to the outer diameter of the second handling tube 2003 at the point of connection to the corresponding holding device 2007. In this embodiment, the gear ratio of the gearbox 2020 preferably causes the two handling tubes 2002, 2003 to rotate at the same speed (in opposite directions), so that the wire structure 2006 (or connectors that connect the wire structure 2006 to the corresponding holding devices 2007) wind around, or unwind from, the corresponding handling tubes 2002, 2003 at the same rate. However, in other embodiments, the outer diameter of the first handling tube 2002 may be larger or smaller than the outer diameter of the second handling tube 2003 at their points of connection to the holding devices 2007. For example, because the second handling tube 2003 traverses through the interior of the first handling tube 2002, at least some portions of the second handling tube 2003 will have an outer diameter that is smaller than the outer diameter of the first handling tube 2002. If, at the points of connection to the holding devices 2007, the outer diameters of the handling tubes 2002, 2003 are not equal, the gearbox 2020 may include a gear ratio that causes the two handling tubes 2002, 2003 to rotate at different rates. For example, the handling tube with the larger outer diameter will wind more length of a connector around the tube as it undergoes a single revolution compared to the handling tube with the smaller outer diameter. Because it is desirable for the connectors to wind around their respective tubes at the same rate (e.g. length of winding per unit time), the gear ratio may be set so that the tangential speed of each handling tube is similar or identical. In other words, the smaller tube will need to revolve at a greater angular rate than the larger tube in order to wind the same length of connector around the tubes per unit time. This concept applies to the embodiments described below.

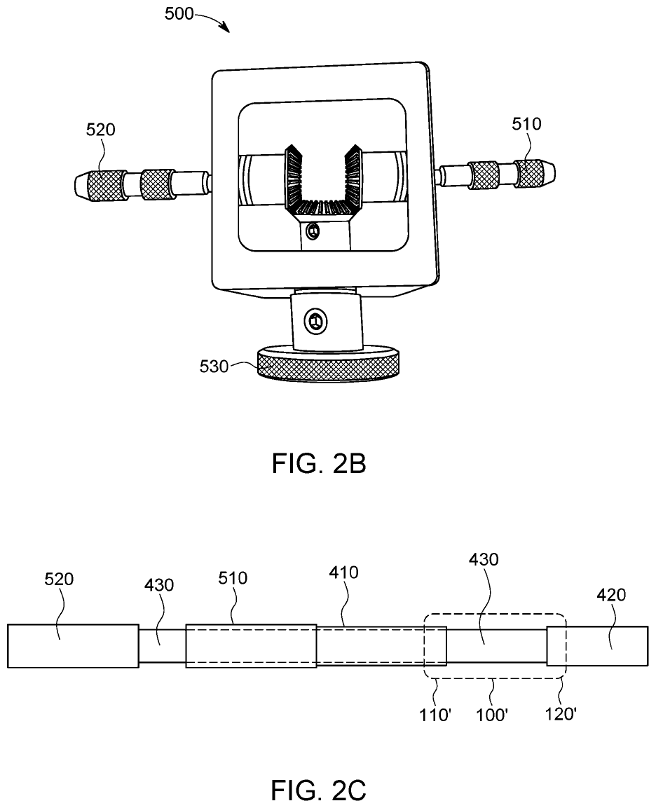

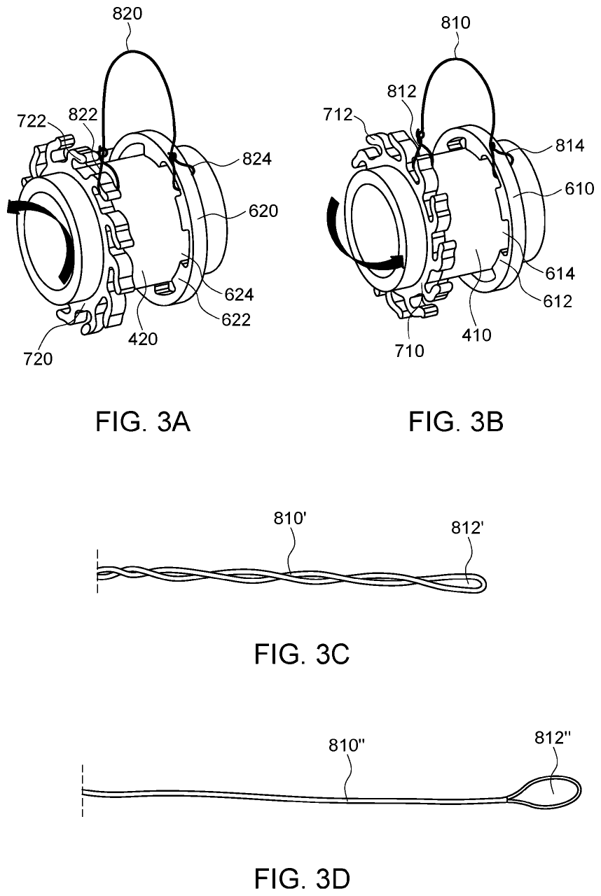

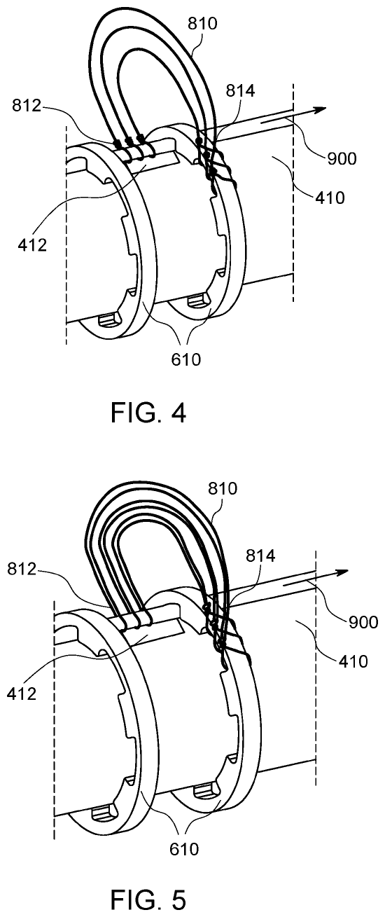

FIG. 2A illustrates a frame 100′ of a prosthetic heart valve in a state of partial expansion having a peanut-shape. Frame 100′ may be substantially similar to frame 100, although some particular differences may exist. Referring to FIG. 2A, the frame 100′ includes an atrial end 110′ which may be substantially similar to atrial end 110, a ventricular portion 120′ substantially similar to ventricular portion 120, and a central portion 130′ substantially similar to central portion 130. A commissure support 135′ similar or identical to commissure support 300 may be included on the frame 100′. In this partially expanded state, an inflow end portion of the atrial disk 110′ is maintained in a fully or nearly-fully collapsed condition via a suture connection to a shaft system 400 of the delivery device, and an outflow end portion of the ventricular disk 120′ is maintained in a fully or nearly-fully collapsed condition via a suture connection to the shaft system 400. The shaft system 400 may be similar or identical to the tubular assembly 2001 described above. For example, the shaft system 400 may include an atrial tube 410 that is similar or identical to first handling tube 2002, and a center rod 430 and ventricular tube 420 that, together, are similar or identical to the second handling tube 2003.

For example, still referring to FIG. 2A, the shaft system 400 may include an atrial tube 410 that has a first end that terminates at or near the atrial disk 110′, and a second opposite end coupled, in a rotationally fixed manner, to a component of a gear system such as gearbox 2020 of control interface 2008. FIG. 2B shows a specific gear system 500 mainly intended to conceptually illustrate one type of system that may produce the desired rotation. For example, the second opposite end of the atrial tube 410 may be rotationally fixes relative to a first pin vise 510 of the gear system 500. FIG. 2B shows the gear system 500 disconnected from any other component, but it should be understood that a conceptually similar device may replace the gearbox 2020 shown in FIG. 1D. Referring back to FIG. 2A, a center rod 430 that has a smaller diameter than the atrial tube 410 passes through the center of the atrial tube 410. The center rod 430 has a first end rotationally fixed (e.g. via laser welding) to a ventricular tube 420, which may have a similar diameter to the atrial tube 410. The center rod 430 and ventricular tube 420 together may be similar or identical to the second handling tube 2003 of FIG. 1E. In other words, although atrial tube 410, ventricular tube 420, and center rod 430 are generally illustrated as rigid rods, they are preferably flexible catheter members as described in connection with tubular assembly 2001. The center rod 430 may pass through the atrial tube 410, through a box of the gear system 500, where a second end of the center rod 430 is coupled, in a rotationally fixed manner, to a second pin vise 520 of the gear system 500. As with the first pin vise 510, the second pin vise 520 just represents one example of a fixed connection, for example between the second handling tube 2003 to the gearbox 2020 of FIG. 1D.

Referring now to FIG. 2B, each pin vise 510, 520 may have a geared end that meshes with a geared end of a knob 530. The geared ends intermesh together such that, upon rotation of the knob 530 in a first direction, the gears interact to rotate the two pin vises 510, 520 in opposite directions than each other. The knob 530 may be an example of a manual operating control 2021 of the control interface 2008. As with the pin vises 510, 520 representing particular examples of fixed connectors, the knob 530 represents a particular type of a manual operating control 2021. FIG. 2C is a highly schematic view of the connection between the second pin vise 520 and the center rod 430, the connection between the center rod 430 and the ventricular tube 420, and the connection between the atrial tube 410 and the first pin vise 510, with other components of the gear system 500 being omitted. The frame 100′, as well as the connection between the atrial disk 110′ and the first end of the atrial tube 410, and the connection between an end of the ventricular tube 420 and the ventricular disk 120′, are schematically shown in FIG. 2C. As is described below in greater detail, by rotating knob 530 in one direction, the atrial tube 410 will rotate in a first direction at its connection to the atrial disk 110′, while the center rod 430 and the ventricular tube 420 will rotate in a second direction (opposite to the first direction) at the connection to the ventricular disk 120′. It should be understood the shaft system 400 and gear system 500 are mere examples of the control interface 2008 and tubular assembly 1, and it should be understood that the embodiments described herein should be generally thought of as modifications that can be made to the tubular instrument 2000. As noted above, one mechanism that tubular instrument 2000 lacks is the ability to release the prosthetic heart valve (or wire structure 2006) easily and precisely from the tubular instrument 2000. However, it should be understood that various different mechanisms may be used in conjunction with the oppositely rotating atrial tube 410 (or first handling tube 2002) and ventricular tube 420 (or second handling tube 2003) to bring (or keep) the atrial disk 110′ and ventricular disk 120′ in close contact with the shaft system 400. Examples of these mechanisms (which should be understood to be examples of the holding devices 2007), and additional mechanisms to allow for full disconnection between the shaft system 400 and the frame 100′, are described in more detail below. In other words, although the various control mechanisms described below are generally described in connection with the shaft system 400, it should be understood that such mechanisms may be used with the tubular assembly 2001, including as specific examples of the holding devices 2007 thereof.

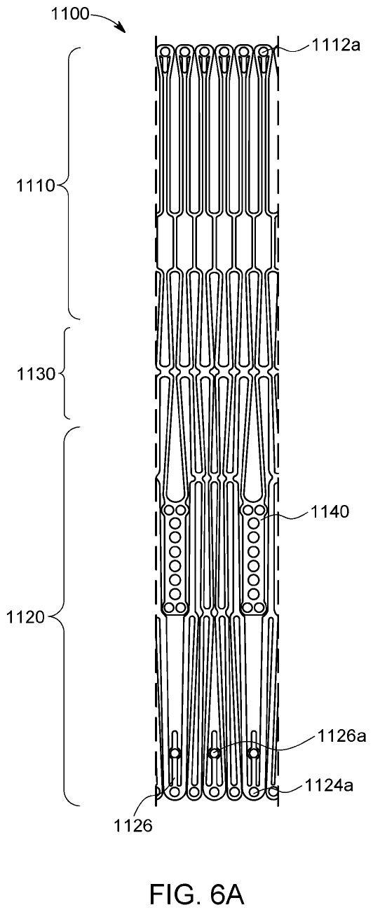

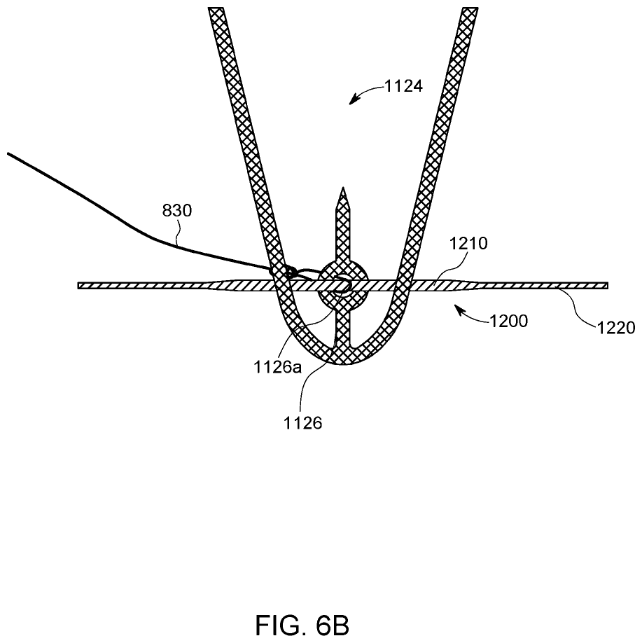

FIG. 3A illustrates a portion of the ventricular tube 420 and FIG. 3B illustrates a portion of the atrial tube 410. Each tube 410, 420 may include a mounting ring 610, 620 fixedly connected to the respective tube 410, 420. Generally, the various rings described herein are described as being “fixed” to the respective tube, but it should be understood that this encompasses a situation in which the ring is integrally formed with the respective tube. Each mounting ring 610, 620 is preferably fixed to its associated tube 410, 420 so that no movement (axial or rotational) can occur between each mounting ring 610, 620 and its associated tube 410, 420. For example, each mounting ring 610, 620 may be laser welded to its associated tube 410, 420. Each mounting ring 610, 620 may be similar or identical, and may include a generally circular or cylindrical outer surface, with inward projections 612, 622 alternating with recesses or notches 614, 624. Each notch 614, 624 may be positioned between a pair of circumferentially adjacent protrusions 612, 622, and each protrusion 612, 622 may be positioned between a pair of circumferentially adjacent notches 614, 624. The protrusions 612, 622 may be in direct contact with an outer surface of the associated tube 410, 420, with the notches 614, 624 creating a void space between the outside of the tube 410, 420 and the interior surface of the mounting ring 610, 620. With this configuration, each notch 614, 624 may serve as a location for a first end 814, 824 of an associated suture 810, 820 to attach to the mounting ring 610, 620. Although the term suture is used, it should be understood that other string or wire-like devices may be used in place of sutures. As shown in FIGS. 3A-B, the first end 814, 824 of each suture 810, 820 may be fixed to the associated mounting ring 610, 620 at notches 614, 624 via knots or any other securement mechanism that ensures the first ends 814, 824 of the sutures 810, 820 cannot disconnect from the mounting ring 610, 620. The positioning of each notch 614, 624 between adjacent protrusions 612, 622 also helps ensure that the first end 814, 824 of each suture 810, 820 cannot slip a significant distance around the circumference of the mounting rings 610, 620, so that the first end 814, 824 of each suture is confined to the area of the notch 614, 624. Although only one suture 810, 820 is shown in FIGS. 3A-B, it should be understood that a plurality of sutures may be provided on each mounting ring 610, 620 with one or more sutures per notch 614, 624. It should be understood that, although the term “suture” is used throughout in connection with sutures 810, 820, and related components, the term “suture” in these contexts encompasses cables, wires, or other strand-like members.

Still referring to FIGS. 3A-B, each tube 410, 420 may include a suture ring 710, 720 fixedly connected to the respective tube 410, 420. Each suture ring 710, 720 is preferably fixed to its associated tube 410, 420 so that no movement (axial or rotational) can occur between each suture ring 710, 720 and its associated tube 410, 420. For example, each suture ring 710, 720 may be laser welded to its associated tube 410, 420. Each suture ring 710, 720 may be similar or identical, but may be mounted to their associated tubes 410, 420 in opposite rotational orientations. Each suture ring 710, 720 may include a generally circular or cylindrical inner surface in direct contact with the outer surface of the associated tube 410, 420. A plurality of teeth or fingers 712, 722 may extend from each suture ring 710, 720 with each finger 712, 722 having a general “L”-shape with a first portion extending radially outward from the center of the associated ring 710, 720 and a second portion that extends in a generally circumferential direction about a central axis passing through the ring 710, 720. The second portion of each finger 712, 722 terminates in a free end, and the different rotational mounting configurations of the two suture rings 710, 720 results in each free end of each finger 712 of ring 710 facing in an anticlockwise direction and each free end of each finger 722 of ring 720 facing in a clockwise direction. It should be understood that the clockwise and anticlockwise directionalities are just conventions, with the important feature being that all of the free ends of the fingers 712 follow one orientation around the circumference of the ring 710, while all of the free ends of the fingers 722 follow another opposite orientation around the circumference of the ring 720. With the configuration described above, each finger 712, 722 may serve as a location for a second looped end 812, 822 or eyelet of an associated suture 810, 820 to attach to the suture ring 710, 720 by slipping over a finger 712, 722. When each suture ring 710, 720 rotates in a direction where the free end of each finger 712, 722 points in or leads in the direction of rotation, the suture eyelets 812, 822 will tend to remain connected to the associated finger 712, 722. When each suture ring 710, 720 rotates in the opposition direction away from where the free end of each finger 712, 722 points, so that the free ends trail (instead of lead) the rotation, the suture eyelets 812, 822 will tend to slip off the finger 712, 722 and become disconnected from the suture ring 710, 720.

Still referring to FIGS. 3A-B, each suture 810, 820 has a central portion extending between the first end 814, 824 and the eyelet or second looped end 812, 822. In use, the frame 100′ may be positioned so that the atrial disk 110′ is positioned over or adjacent to the atrial mounting ring 610 and suture ring 710, and the ventricular disk 120′ is positioned over or adjacent to the ventricular mounting ring 620 and suture ring 720. With this positioning, the center portion of each suture 810 may loop through the interior of a cell in the atrial disk 110′ (e.g. at or near the inflow-most apex) and the center portion of each suture 820 may loop through the interior of a cell in the ventricular disk 120′ (e.g. at or near the outflow-most apex). For example, if frame 100′ has the same structure as frame 100, the atrial sutures may loop through the atrial cells 112, and the ventricular sutures may loop through the ventricular cells 124b, 124c. With this configuration, as long as the eyelets 812, 822 of the sutures 810, 820 remain connected to the associated fingers 712, 722, the frame 100′ is maintained connected to the associated tube 410, 420. When the knob 530 is rotated in one direction, the tube 410 and suture ring 710 rotate in a first direction while the tube 420 and suture ring 720 rotate in the opposite direction, causing the associated sutures 810, 820 to pull the atrial disk 110′ and ventricular disk 120′ toward the associated tubes 410, 420, either forcing the ends of the frame 100′ to collapse, or maintaining the ends of the frame 100′ in a collapsed state, so that the frame 100′ takes the “peanut” shape in FIG. 2A. When the prosthetic heart valve is in the desired position relative to the native valve annulus, the knob 530 is rotated in the opposite direction, releasing tension on the sutures 810, 820, allowing the ends of the frame 100′ to expand, and eventually allowing the eyelets 812, 822 of the sutures 810, 820 to slip off their associated fingers 712, 722 to fully release the frame 100′ (and thus the overall prosthetic heart valve) from the delivery shaft system 400. It should be understood that, by having the tubes 410, 420 rotate in opposite directions, the sutures 810, 820 more effectively are able to pull the frame 100′ toward the shaft system 400 as the sutures 810, 820 wind (in opposite directions) around their associated tubes 410, 420.

Although the sutures 810, 820 of FIGS. 3A-3B are shown with an eyelet 812, 822 formed by simply creating a knot at the end of the suture, eyelets or loops may be formed in other ways. For example, as shown in FIG. 3C, any of the sutures 810′ may be formed as a suture or string that is wound around a center line to form an eyelet 812′ at its distal end. In other embodiments, as shown in FIG. 3D, any of the sutures 810″ may be woven in a manner to create an eyelet 812″ at its distal end. For example, suture 810″ has a main body portion which is larger than the suture forming the eyelet 812″ so that there is a smooth transition between the main body portion and the eyelet 812″. When a knot is used to form an eyelet, such as the knots shown in FIGS. 3A-B for eyelets 812, 822, there is a risk that the knot may interfere with the stent or frame structure (such as a cell apex), or if the suture is threaded through a hole (as described in other embodiments below), the knot may get caught within such a hole. It should be understood that any of eyelet options may be suitable for use.

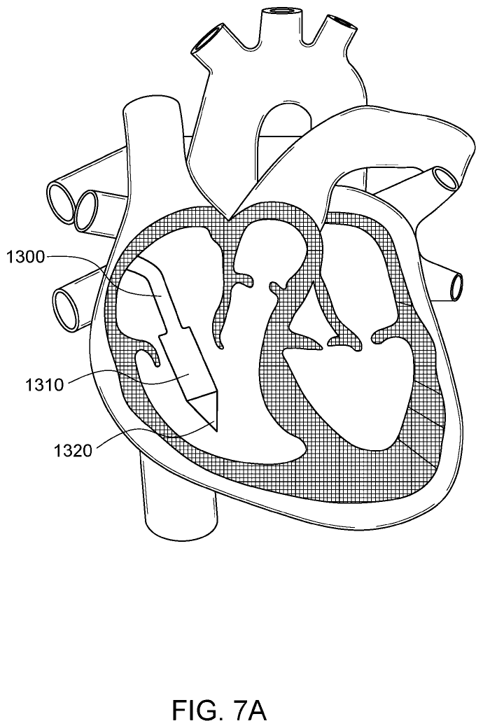

FIG. 4 illustrates an alternate system for controlling the deployment of the frame 100′. FIG. 4 illustrates the atrial tube 410, but it should be understood that an identical system may also be provided on the ventricular tube 420. As shown in FIG. 4, the atrial tube 410 includes two mounting rings 610 spaced apart from each other, each mounting ring 610 being similar or identical to the mounting ring 610 of FIG. 3A. One or more sutures 810 may be fixed to the mounting ring 610 in the same way as shown and described in FIGS. 3A-B. However, instead of the eyelets 812 of the sutures 810 being connected to a suture ring as in FIGS. 3A-B, the eyelets 812 may all loop over a single pull rod 900. As shown in FIG. 4, the pull rod 900 may pass through the notch of one of the mounting rings 610 and through a notch of the other mounting ring 610 so that the eyelets 812 looped over the pull rod 900 are unable to slip off the pull rod 900 as long as the pull rod 900 extends between both mounting rings 610. The atrial tube 412 may include a notch or cutout 412 to provide space for the eyelets 812 of the sutures 810 between the pull rod 900 and the atrial tube 410. The ventricular tube may similarly include a pair of mounting rings, a notch, and a separate pull rod. As with the embodiment of FIGS. 3A-D, the sutures 810 on the atrial tube 410 may pass through the atrial disk 110′ while the sutures on the ventricular tube may pass through the ventricular disk 120′. Turning the knob 530 will turn the atrial and ventricular tubes 410, 420 in opposite directions to wind the sutures around the tube (or unwind the sutures depending on the direction of rotation). Once the user is ready to fully release the frame 100′ from the shaft system 400, the pull rod 900 may be pulled proximally until the eyelets 812 slip off the distal end of the pull rod 900. Although eyelets 812 are shown as being formed by knots, it should be understood that the other suture loop alternatives described above may apply to this embodiment as well.

FIG. 5 illustrates a slightly different alternative embodiment compared to FIG. 4. The only difference between the embodiment of FIG. 5 and FIG. 4 is that the sutures 810 do not terminate in an eyelet 812 that is looped over the pull rod 900. Rather, each suture 810 has a first knotted end coupled to the mounting ring 610, with the center portion of the suture 810 looping around the pull rod 900, and the second end of the suture forming another knot fixed to the mounting ring 610 next to the first knotted end. Other than the way in which the ends of the sutures 810 of FIG. 5 are secured to the mounting ring 610 and pull rod 900, the structure and function of the embodiment of FIG. 5 are identical to that of FIG. 4. In some examples, the pull rod 900 may be connected to a flexible suture which is wound around the rotating shaft. The number of times the suture is wound around the shaft may be similar to the number of rotations needed to open the ventricular and atrial disk. After the disk is fully open the suture which is connected to the rod 900 may be pulled back and thereby release the suture loop as described above.

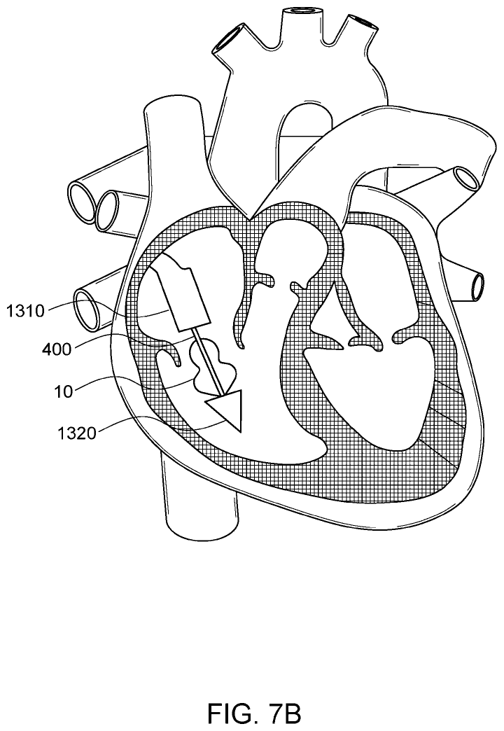

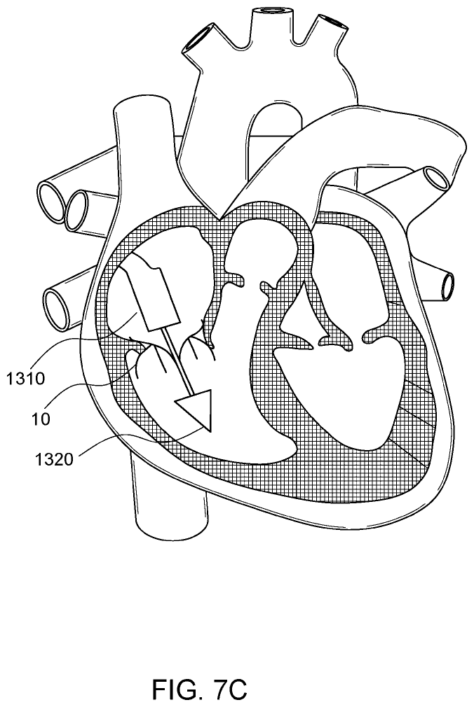

Although the embodiments described above generally rely on a suture passing through a cell of the frame 100 or 100′, in other embodiments, the frame may include a dedicated feature to connect to the sutures. For example, FIG. 6A illustrates a portion of a cut pattern of a frame 1100 that is generally similar to frame 100 and which can be used with prosthetic heart valve 10 and/or in place of frame 100′. As with frames 100 and 100′, frame 1100 may include an atrial disk 1110, a ventricular disk 1120, and a central waist 1130. In the expanded condition, frame 1100 may have a shape similar to that shown in FIG. 1C. The description of frame 100 generally applies to frame 1100, except for the differences described below. Thus, only the differences will be described for the sake of brevity. One difference in frame 1100 is that the CAFs 1140 have a generally dog-boned shape with a single column of eyelets and a pair of side-by-side eyelets on either end of the column. This structure may assist with coupling to the commissure support 300, as is described in more detail in U.S. Provisional Patent Application No. 63/384,521, filed Nov. 21, 2022, and titled “Transcatheter Prosthetic Atrioventricular Valve with Stiffening Structure,” the disclosure of which is hereby incorporated by reference herein. Another difference is that the atrial cells may include an aperture 1112a formed in the tip of the atrial cell, having a size configured to accept a suture therethrough. Similarly, apertures 1124a may be formed in the tips of the ventricular cells, and for any tines provided in the ventricular cells, apertures 1126a may be provided in those tines. Apertures 1112a may be used to receive a center portion of the atrial sutures 810 therethrough, while apertures 1124a may be used to receive a center portion of the ventricular sutures 820 therethrough. It may be preferable, but it is not necessary, to use sutures similar to suture 810′ or 810″ that include eyelets 812′ or 812″ without knots, as those knots could interfere with the sutures pulling through the apertures. Thus, by using the frame 1100 of FIG. 6A with prosthetic heart valve 10, the peanut shape shown in FIG. 2A may be achieved with atrial sutures 810 connecting the atrial disk 1110, via apertures 1112a, to the atrial tube 410, and with ventricular sutures 820 connecting the ventricular disk 1120, via apertures 1124a, to the ventricular tube 420, and rotating the atrial tube 410 and ventricular tube 420 in opposite directions using gear system 500. It should be understood that any suitable expansion restriction mechanisms, including those shown in FIGS. 3A-B, 4, and 5 may be used to achieve the expansion control, as well as the controlled release of the frame 1100 from the shaft system 400 when the prosthetic heart valve 10 is confirmed to be in the desired position.

One potential issue with allowing the prosthetic heart valve 10 (and particularly the frame 100, 100′, 1100 thereof) to achieve the peanut shape of FIG. 2A, is that the tines (e.g. tines 126 or 1126) may tend to protrude radially outwardly as they take their set shape, even while the frame is otherwise constrained to this peanut shape. These outwardly extending tines 126, 1126 might end up interfering with native tissue, for example by engaging or becoming entangled with the sub-valvular apparatus, such as the chordae tendineae. This potential problem may be avoided by utilizing the apertures 1126a provide in the tines. For example, FIG. 6B shows an enlarged view of one of the ventricular cells 1124 of frame 1100 which includes a tine 1126 with an aperture 1126a. As shown in FIG. 6B, a tine suture 830 may have an end terminating in an eyelet (including a knot or excluding a knot like the embodiments of FIGS. 3C-D), and that eyelet may at least partially extend through the aperture 1126a to a radially outer surface of the frame 1100. The opposite end of the tine suture 830 may be coupled to the ventricular expansion control mechanism (e.g. any of the mounting rings 610 that are shown in FIG. 3A, or the ventricular versions of the embodiments of FIGS. 4-5). A separate wire 1200 may pass through the eyelet of the suture 830 on the radially-outer surface of the tine 1126, but may extend across the struts forming the apex of the ventricular cell 1124 on the radially-interior surface of the cell 1124. The wire 1200 may be a flexible wire, for example formed of Nitinol or other similar materials, and in some embodiments may include a relatively thick portion 1210 to provide maximum support at the points of contact with the frame 1100, but relatively thin portions 1220 (e.g. by being ground down) away from the points of contact to provide maximum flexibility in areas of non-contact. Instead of a thick and thin portion, the wire may include a metal portion in place of the thick portion 1220, and a suture (or suture-like) material in place of the thin portions 1220, with the suture connected to the metal portion by any suitable mechanism, including by swaging, or similar to other ways in which sutures are typically connected to needles. For example, the metal portion could be a Nitinol tube which is swaged on each end to a suture or suture portion. In other examples, the wire 1200 could be formed as a monofilament suture that is thinned out (e.g. stretched) so that it has thicker sections (not thinned or stretched) where desired and thinner sections where desired. Regardless of the specific way in which the wire 1200 is formed, while tension is maintained on the tine suture 830, the wire 1200 pulls the tine 1126 radially inwardly so that it will not interfere with the sub-valvular apparatus. When the position of the prosthetic heart valve 10 is confirmed while the frame 1100 is still in the controlled peanut-shape shown in FIG. 2A, the wire 1200 may be withdrawn such that the tine suture 830 is no longer connected to the tine 1126, allowing the tine 1126 to take it set-shape (in which it points radially outwardly) and engage the native tissue as the prosthetic heart valve 10 expands into its final deployed configuration.

Although only a portion of the flexible wire 1200 is shown in FIG. 6B, it should be understood that the flexible wire is longer than shown and may form a loop around the circumference of the frame 1100 so that each tine 1126 may be controlled by the single wire 1200, with ends of the wire 1200 extending through channels of the delivery system to maintained a closed loop for wire 1200. When the user is ready to disconnected the wire 1200 from the tine sutures 830 to allow for the tines 1126 to move outwardly, one end of the wire 1200 may be pulled proximally from the delivery system until the other end pulls through all of the eyelets of the sutures 830.

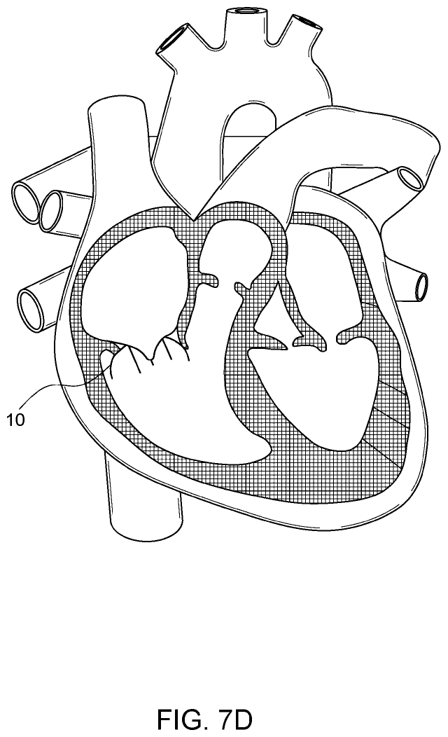

FIGS. 7A-7D illustrate steps of an exemplary delivery of prosthetic heart valve 10 using the expansion control mechanisms described herein. Prior to delivery, the prosthetic heart valve 10 is loaded in a collapsed condition within a valve cover 1310 of a delivery catheter 1300. As part of this loading process, the prosthetic heart valve 10 may be positioned over the shaft system 400 (or over the tubular assembly 2001) and atrial sutures 810 passed through the atrial disk 1110 (either through cells or apertures 1112a) and ventricular sutures 820 passed through the ventricular disk 1120 (either through cells or apertures 1124a). Any of the expansion restriction mechanisms described above may be used (e.g. in place of holding devices 2007), and the gear system 500 (or control interface 2008) may be activated to wind the sutures 810, 820 around the respective tube (e.g. handling tube 2002, 2003) to draw the atrial disk 1110 and ventricular disk 1120 toward their respective tubes. If tine sutures 830 are used, they may be positioned through the tine apertures 1126 and the wire 1200 may be threaded through the eyelets to form a closed loop that can maintain the tines 1126 in an inward position.

Referring to FIG. 7A, after the prosthetic heart valve 10 is collapsed within the valve cover 1310 and the various control sutures are in place, the delivery catheter 1300 may be advanced into the patient, for example through the femoral vein, with a nosecone 1320 leading the advancement. The delivery catheter 1300 may enter the right atrium via the inferior vena cava and, with the assistance of steering controls, the valve cover 1310 may be positioned through the native tricuspid valve.

Once the valve cover 1310 is in the desired position, the valve cover 1310 may be withdrawn, allowing the prosthetic heart valve 10 to start to self-expand. However, because the atrial sutures 810 and ventricular sutures 820 are wound around their associated tubes of the shaft system 400, the atrial disk and ventricular disk are restricted from expanding. As shown in FIG. 7B, this may result in the prosthetic heart valve 10 expanding to have a peanut shape. Further, as noted above, this partial expansion shortens the axial extent of the prosthetic heart valve 10 (compared to the fully crimped condition), and thus the delivery catheter 1300 does not need to extend as far into the right ventricle as might otherwise be required. While the prosthetic heart valve 10 is in this partially-expanded state, with valve cover 1310 withdrawn, the user may confirm that the prosthetic heart valve 10 is in the desired position for full expansion. If the prosthetic heart valve 10 is not in the desired position for full expansion, the position and/or orientation may be modified until the user is satisfied. It should also be understood that, if the tine sutures 830 are implemented in the method, while the prosthetic heart valve 10 is in the condition shown in FIG. 7B, the tines 1126 are restricted from pointing outwardly and engaging or otherwise becoming entangled with the sub-valvular apparatus.

Even if the prosthetic heart valve 10 is positioned too deep into the ventricle, a correction during expansion of the prosthetic heart valve 10 should be simple to achieve. For example, when unwinding the sutures from the respective tube, the prosthetic heart valve 10 will foreshorten (axially) both at the atrial disk and the ventricular disk. While this foreshortening is occurring, the prosthetic heart valve 10 can be further pulled back (toward the atrium). Desirable, due to the simultaneous expansion of the ventricular disk and atrial disk during the unwinding (and eventual release), the prosthetic heart valve 10 tends to self-center itself. Although self-centering has been known for sequential deployment (e.g. ventricular deployment, followed by atrial deployment), the simultaneous deployment allowed by the systems described herein may result in significantly better self-centering. Still further, it should be understood that in the “peanut” shape, the actual valve leaflets (or valve assembly) are not collapsed, or are only slightly collapsed. As a result, at the moment the atrial disk and ventricular disk are partially open, the prosthetic valve leaflets are already functioning. This is in contrast to known prosthetic heart valves in which the prosthetic heart valve is sequentially released, and the prosthetic leaflets do not start functioning until after the final deployment of the atrial disk.

Once the user confirms that the prosthetic heart valve 10 is in the desired position, the user may allow the prosthetic heart valve 10 to fully expand. To achieve this full expansion, the user may rotate the knob 530 of the gear system 500 to allow the atrial sutures 810 and ventricular sutures 820 to unwind from the shaft system 400 such that the tension on the prosthetic heart valve 10 caused by the atrial sutures 810 and ventricular sutures 820 is released. Further, as this tension is released, the atrial sutures 810 and ventricular sutures 820 will disconnect from the prosthetic heart valve 10. If the expansion control mechanism of FIGS. 3A-B is used, the suture eyelets will slip off the fingers of the suture rings. If the expansion control mechanism of FIG. 4 is used, the pull rod 900 will be pulled proximally allowing the suture eyelets to slip off the pull rod 900. If the expansion control mechanism of FIG. 5 is used, as the pull rod 900 is pulled proximally, the middle portion of the sutures will detach from the pull rod 900 and pull through the prosthetic heart valve 10. Just prior to, just after, or simultaneous with the release of atrial sutures 810 and ventricular sutures 820, the user may withdraw the flexible wire 1200 proximally through the delivery catheter 1300 if such a flexible wire 1200 is used. This will allow the tines 1126 to move outwardly and frictionally engage tissue to assist with anchoring. The fully released condition is shown in FIG. 7C.

After complete release of the prosthetic heart valve 10, the shaft system 400 and nosecone 1320 may be withdrawn through the deployed prosthetic heart valve 10 (for example until the nosecone 1310 abuts the distal end of the valve cover 1310), and the delivery catheter 1300 may be removed from the patient. At this point, the prosthetic heart valve 10 is fully deployed, as shown in FIG. 7D, and the procedure may be completed.

Although the disclosure herein is generally described in the context of prosthetic tricuspid valve delivery and deployment, it should be understood that similar or identical features may be used in a prosthetic mitral valve, or even in prosthetic aortic or pulmonary valves. Although various rings are described herein as pairs of rings (e.g., rings 610 and 710 as a pair, rings 620 and 720 as a pair, two rings 610 as a pair, etc.), it should be understood that, in some examples, these pairs of rings may be provided as a single ring having features of both individual ring of the pair of rings. For example, in some embodiments, rings 620 and 720 may be provided as a single member having the projections 622 and notches 624 of ring 620 as well as the fingers 722 of ring 720. Other pairs of rings described herein may be similarly provided as a single combined ring where appropriate.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve system, comprising:
a prosthetic heart valve for replacing a native atrioventricular valve, the prosthetic heart valve including:
a collapsible and expandable frame, the frame including an atrial disk, a ventricular disk, and a center portion extending between the atrial disk and the ventricular disk, and a plurality of prosthetic leaflets disposed within the frame; and
a delivery device for delivering and deploying the prosthetic heart valve, the delivery device including:
a catheter having a valve cover at a distal end thereof, the valve cover configured to maintain the prosthetic heart valve in a collapsed condition for delivery; and
an expansion restriction mechanism and a shaft system including an atrial tube and a ventricular tube, the expansion restriction mechanism including a first pair of rings fixed to the atrial tube and a second pair of rings fixed to the ventricular tube,
wherein in a delivery condition of the system, the prosthetic heart valve is collapsed within the valve cover with the atrial disk positioned adjacent to the atrial tube and the ventricular disk positioned adjacent to the ventricular tube, and a plurality of first sutures connect the first pair of rings and extend through the atrial disk, and a plurality of second sutures connect the second pair of rings and extend through the ventricular disk.

2. The prosthetic heart valve system of claim 1, wherein in a partially deployed condition of the system:
the valve cover is withdrawn relative to the prosthetic heart valve so that the valve cover does not restrict expansion of the prosthetic heart valve;
the plurality of first sutures are wound around the atrial tube so that an inflow portion of the atrial disk is restricted from self-expanding;
the plurality of second sutures are wound around the ventricular tube so that an outflow portion of the ventricular disk is restricted from self-expanding; and
an outflow portion of the atrial disk, an inflow portion of the ventricular disk, and the center portion of the frame are all at least partially expanded.

3. The prosthetic heart valve system of claim 2, wherein in the partially deployed condition of the system, the prosthetic heart valve has a peanut shape in which the at least partially expanded inflow portion of the ventricular disk has a larger diameter than the at least partially expanded outflow portion of the atrial disk, which has a larger diameter than the at least partially expended center portion of the frame.

4. The prosthetic heart valve system of claim 2, further comprising a gear system coupled to the delivery device, the gear system having a first connector coupled to the atrial tube, a second connector coupled to the ventricular tube, and a knob operatively coupled to the first and second connectors such that rotation of the knob in a first rotation direction first and second connectors, and thus the atrial and ventricular tubes, to simultaneously rotate in opposite directions.

5. The prosthetic heart valve system of claim 4, wherein the first connector is directly coupled to the atrial tube, and the second connector is directly connected to a central rod which passes through the atrial tube and directly connects to the ventricular tube.

6. The prosthetic heart valve system of claim 1, wherein the first pair of rings includes a first mounting ring and a first suture ring, and the second pair of rings includes a second mounting ring and a second suture ring, the first plurality of sutures being fixedly coupled to the first mounting ring and releasably coupled to the first suture ring, and the second plurality of sutures being fixedly coupled to the second mounting ring and releasably coupled to the second suture ring.

7. The prosthetic heart valve system of claim 6, wherein the first suture ring has a plurality of first fingers that each have a free end pointing in a first rotational direction, and the second suture ring has a plurality of second fingers that each have a free end pointing in a second rotational direction opposite the first rotational direction.

8. The prosthetic heart valve system of claim 1, wherein the first pair of rings includes first and second mounting rings, and the second pair of rings including third and fourth mounting rings, the first plurality of sutures being fixedly coupled to both the first and second mounting rings, and the second plurality of sutures being fixedly coupled to both the third and fourth mounting rings.

9. The prosthetic heart valve system of claim 8, further comprising a first pull rod extending through the first pair of rings, eyelets of the first plurality of sutures receiving the first pull rod therethrough in the delivery condition of the system, and a second pull rod extending through the second pair of rings, eyelets of the second plurality of sutures receiving the second pull rod therethrough in the delivery condition of the system, the first and second pull rods each being configured to be withdrawn relative to the respective pair of rings to release the respective eyelets from the respective pull rod.

10. The prosthetic heart valve system of claim 1, wherein the atrial disk includes a plurality of atrial apertures formed at respective inflow tips of the atrial disk, and the ventricular disk includes a plurality of apertures formed at respective outflow tips of the ventricular disk, and in the delivery condition of the system, the plurality of first sutures pass through respective ones of the atrial apertures, and the plurality of second sutures pass through respective ones of the ventricular apertures.

11. A method of implanting a prosthetic heart valve, the method comprising:
loading the prosthetic heart valve into a delivery device, the prosthetic heart valve including a collapsible and expandable frame having an atrial disk, a ventricular disk, a center portion extending between the atrial disk and the ventricular disk, and a plurality of prosthetic leaflets disposed within the frame;
advancing the delivery device to a native heart valve of a patient while the prosthetic heart valve is maintained in a collapsed condition by a valve cover of the delivery device;
while the delivery device is positioned in or adjacent to the native heart valve, starting to deploy the prosthetic heart valve by withdrawing the valve cover so that the frame begins to self-expand;
wherein as the frame begins to self-expand, an outflow end portion of the ventricular disk is restricted from self-expanding due to a connection between the outflow end portion of the ventricular disk and a ventricular tube positioned inside the frame, such that the ventricular disk is partially expanded after the valve cover is withdrawn;
after the valve cover is withdrawn and the ventricular disk is partially expanded, confirming a desired position of the prosthetic heart valve relative to the native heart valve; and
after confirming the desired position of the prosthetic heart valve relative to the native heart valve, releasing the connection between the outflow end portion of the ventricular disk and the ventricular tube to allow the ventricular disk to expand into engagement with the native heart valve.

12. The method of claim 11, wherein as the frame begins to self-expand, an inflow end portion of the atrial disk is restricted from self-expanding due to a connection between the inflow end portion of the atrial disk and an atrial tube positioned inside the frame, such that the atrial disk is partially expanded after the valve cover is withdrawn.

13. The method of claim 12, wherein after the valve cover is withdrawn, but while the connection between the outflow end portion of the ventricular disk and the ventricular tube is maintained and while the connection between the inflow end portion of the atrial disk and the atrial tube is maintained, the prosthetic heart valve has a peanut shape in which an outflow portion of the atrial disk is at least partially expanded and an inflow portion of the ventricular disk is at least partially expanded.

14. The method of claim 13, wherein when the prosthetic heart valve has the peanut shape, the at least partially expanded atrial disk has a first diameter, the at least partially expanded ventricular disk has a second diameter, and the center portion of the frame has a third diameter, the second diameter being larger than the first diameter, and the first diameter being larger than the third diameter.

15. The method of claim 12, wherein, after confirming the desired position of the prosthetic heart valve relative to the native heart valve, the connection between the inflow end portion of the atrial disk and the atrial tube is released to allow the atrial disk to expand into engagement with the native heart valve.

16. The method of claim 15, wherein the connection between the outflow end portion of the ventricular disk and the ventricular tube is formed by a first plurality of sutures, and the connection between the inflow end portion of the atrial disk and the atrial tube is formed by a second plurality of sutures.

17. The method of claim 16, wherein in the partially expanded condition of the ventricular disk, the first plurality of sutures is wound around the ventricular tube, and in the partially expanded condition of the atrial disk, the second plurality of sutures is wound around the atrial tube.

18. The method of claim 17, wherein releasing the connection between the outflow end portion of the ventricular disk and the ventricular tube includes allowing the first plurality of sutures to unwind from the ventricular tube, and releasing the connection between the inflow end portion of the atrial disk and the atrial tube includes allowing the second plurality of sutures to unwind from the atrial tube.

19. The method of claim 18, wherein allowing the first plurality of sutures to unwind from the ventricular tube and allowing the second plurality of sutures to unwind from the atrial tube includes simultaneously rotating the atrial tube in a first rotational direction and rotating the ventricular tube in a second rotational direction opposite the first rotational direction.

20. The method of claim 19, wherein releasing the connection between the outflow end portion of the ventricular disk and the ventricular tube and releasing the connection between the inflow end portion of the atrial disk and the atrial tube includes either:
(i) allowing the first plurality of sutures and the second plurality of sutures to slip off respective connection points to their respective tubes; or
(ii) actively withdrawing a first pull rod to disconnect the first plurality of sutures from the ventricular tube and actively withdrawing a second pull rod to disconnect the second plurality of sutures from the atrial tube.
